(19) United States
(12) Patent Application Publication
(10) Pub. No.: US 2016/0129182 A1
(43) Pub. Date: May 12, 2016

(54) VIBRATION SENSOR BASED DRUG DELIVERY MONITOR

(71) Applicant: OSCILLARI LLC, Bolinas, CA (US)

(72) Inventors: Jeffrey A. SCHUSTER, Bolinas, CA (US); Stephen J. FARR, Orinda, CA (US); Reuben HALE, Oakland, CA (US)

(73) Assignee: OSCILLARI LLC, Bolinas, CA (US)

(21) Appl. No.: 14/995,822

(22) Filed: Jan. 14, 2016

Related U.S. Application Data
(63) Continuation-in-part of application No. 14/903,936, filed on Jan. 8, 2016.
Application No. 14/903,936 is a continuation-in-part of application No. PCT/US15/51522, filed on Sep. 22, 2015.
(60) Provisional application No. 61/845,670, filed on Jul. 12, 2013.
(60) Provisional application No. 62/054,043, filed on Sep. 23, 2014.

(51) Int. Cl. A61M 5/168; G06F 19/00; A61M 15/00; A61M 5/142; A61M 5/172

(52) U.S. Cl. CPC A61M 5/16831; A61M 5/14248; A61M 5/172; A61M 15/00; G06F 19/3406; G06F 19/3456; A61M 2205/276; A61M 2205/3375; A61M 2205/3569; A61M 2205/3592; A61M 2205/505; A61M 2205/52; A61M 2205/581; A61M 2205/583; A61M 2205/8206

(57) ABSTRACT

A monitoring system comprising a monitor is disclosed that utilizes a vibration sensor to monitor the occurrence and properties of an event. The monitor does not require disassembly of the device to be monitored, or interfere with the operation of the device to be monitored, because the monitor is affixed to the exterior of a device to be monitored or a component thereof, or is integrated into the design of the device to be monitored. In a preferred embodiment, the device to be monitored is a drug delivery device, most preferably an inhaler or autoinjector. The monitoring system includes a display device such as a smartphone or tablet computer for analyzing data related to the device to be monitored usage and displaying information to a user, patient and/or caregiver before, during, and after a usage event. Preferred embodiment monitor the inhalation flow rate through an inhaler, and the dose delivered by an injector.

### CROSS-REFERENCE

This application is a continuation-in-part application of Ser. No. 14/903,936, filed Jan. 8, 2016 which is a 371 National Phase Application of International Patent Application Serial No. PCT/US2014/046367, filed Jul. 11, 2014 which claims the benefit of priority to provisional patent application Ser. No. 61/845,670, filed Jul. 12, 2013 and this application is a continuation-in-part of International Patent Application Serial No. PCT/US2015/051522, filed Sep. 22, 2015 which claims the benefit of priority to provisional patent application Ser. No. 62/054,043, filed Sep. 23, 2014, which are all incorporated herein by reference in their entirety noting that the current application controls to the extent there is any contradiction with any earlier application and to which applications we claim priority under 35 USC §120.

### FIELD OF THE INVENTION

The present invention relates to methods and devices for the monitoring of events from a device to be monitored, for example a drug delivery device, and displaying data, instructions, and feedback to a patient and/or caregiver.

### BACKGROUND OF THE INVENTION

Many devices exist in the art that would benefit from monitoring. Monitoring may include such things at the time and date of a certain correct or incorrect action, a quality of an action, the correct or incorrect sequence of actions, unexpected and/or dangerious actions, etc. Example of devices that might be monitored include appliances such as refrigerators, televisions, telephones, video games, cash machines, doors, doorbells, turnstiles, gates, vending machines, vehicle driving locations, alarms, motors, and the like. Preferred devices include drug delivery devices. Actions that can be monitored include use, preparation, breakage, break in, entrance, exit, wear, imminent failure, and the like.

Many devices exist in the art for delivering drugs to patient. These devices can range from a simple oral capsule to a complex hospital based system. Many technologies currently exist or are disclosed in the art that allow a patient to self administer drugs. These devices include inhalers, autoinjectors, needle free injectors, pumps including patch pumps and bolus pumps, transdermals, sprays, ocular devices, etc.

Many disease states exist wherein available drugs and delivery systems can efficaciously treat many or most patients, but a significant percentage of the patient population are not properly treated due to improper use, or non-use, of the drugs and delivery systems. Examples of disease often not correctly treated include, but are not limited to asthma, COPD, and diabetes. Untreated asthma or COPD can lead to expensive emergency room visits, changing to expensive drugs, including biotech proteins such as omalizumab, extreme patient discomfort, or death. Similarly, untreated diabetes can lead to emergency room visits, blindness, nerve damage, cardiovascular events, loss of foot or leg, blindness, or death. Thus, there is an unmet medical need for better means of determining that patients are self administering their medications properly.

Many options currently exist for training and coaching of patients to properly treat their disease state, especially in diabetes. The long term effectiveness of these methods can be monitored, for example by monitoring morbidity or HbA1c. Short term effectiveness can be gauged by monitoring blood glucose. However, high or low blood glucose can be due to non-delivery of medication, incorrect dose, incorrect delivery, or excess intake of carbohydrates. Attempts can be made to determine the cause of high or low blood sugar via patient interviews, however, such methods are notoriously unreliable. Similar issues exist in other disease states including but not limited to Asthma and COPD. Thus, there is an unmet medical need for a means for determining the cause undertreatment or in-correct treatment of disease states particularly as regards to treating with a drug delivery device.

Autoinjectors are self contained devices for delivering drugs by injection, either intradermally, subcutaneously, or intramuscularly. Autoinjectors can contain a single dose or multiple doses, may be disposable or refillable, and comprise a self-contained power source such as a spring, compressed gas, batteries, or a combustible or pyrotechnic material. Autoinjectors may contain a hypodermic needle, but also may be needle free, jet type injectors. Autoinjectors are often used for chronic conditions where multiple injections must be given in a home setting, for example diabetes, osteoporosis, growth hormone deficiency, and the like. Preferred autoinjectors are multidose, and preferably are dose titratable, for example insulin pens.

Inhalers are devices that allow delivery of drug to the lung, either for treatment of lung diseases such as asthma, chronic obstructive pulmonary disease (COPD), cystic fibrosis, emphysema, chronic bronchitis, pulmonary hypertension, bronchietasis, or for systemic effect for such indications as diabetes, agitation, pain (such as migraine pain, post operative pain, cancer pain) or others. Preferred inhaled drugs for systemic effect are those that either currently must be delivered by an invasive means such as injection, or benefit from a more rapid onset than can be achieved with other routes of delivery such as oral.

Some drugs are dosed at prescribed dosing intervals, such as once a month, once a week, once a day, two times a day, etc. Others are dosed when symptoms are present. In either case it is useful to monitor the time, date, and quality of delivery events, for example to determine if the patient is complying with the prescribed therapy and instructions, or how often he or she is having symptoms.

Many drug delivery devices require a somewhat complex maneuver to deliver a dose. This is especially true of inhalers, wherein the patient must inhale at a prescribed rate and duration to get optimal delivery. Some require coordination of the dose with the inhalation, although many modern inhalation devices are breath actuated, or in the case of dry powder inhalers, breath dispersed, and do not require this coordination. In either case, it is important that the patient continue inhaling for period after the aerosol is generated to ensure the correct dose is delivered deeply to the lung. Other actions may be required, such as shaking the inhaler, priming the inhaler, advancing the inhaler to the next dose, removing a cover or cap, holding the inhaler in a prescribed orientation, such as level, or conducting a breath hold after the delivery. Thus, there is a value in monitoring parameters of a pulmonary delivery event to determine if the patient is inhaling and conducting other actions in a way that will deliver an optimal dose.

Devices exist for monitoring a disease state, for example in a home setting. Examples include pulmonary function tests such as peak flow and FEV1 meters, blood glucose sensors, and the like. Wireless, for example Bluetooth versions exist that can transmit measured data to a computer, tablet, or smartphone, whereby a record of disease state over time can be displayed. In general, these devices are not capable of also monitoring drug delivery events, and displaying this information together with information related to the disease state.

WO 96/13293 describes a system into which an inhaler can be inserted. A pressure transducer measures the pressure drop in the airflow path and based on a previous calibration, calculates inhalation flow rates and volumes. A microprocessor and on board memory analyze and store the data. A means for triggering the device is provided, which initiates delivery only if a predetermined flow rate is achieved early in the inhalation maneuver. A peak flow meter is also supplied. Pulmonary function data and inhalation profile data are stored with time and date stamp, and can be downloaded by a wired connection for review. The device can supply the user with audio feedback, for example when to next take the drug.

U.S. Pat. No. 7,448,375 describes a device for delivery of insulin by inhalation, wherein better control of insulin delivery is achieved by controlling the volume of air inhaled with the insulin aerosol. Additional features are described, such as a lockout that limits the number of deliveries in a period of time, or a green light that guides the user to inhale at the correct flow rate, said green light transitioning to a red light if the user inhales too rapidly or too slowly.

US 2010/0192948 and similar application WO 2013/043063 disclose a monitor for an asthma inhaler that uses an optical means for monitoring the actuation of the inhaler, and an electronic control module to monitor and store data related to patient usage of the inhaler. An optional audio sensor is included to detect sound associated with movement of the medicament container during delivery of a dose and/or sound associated with the inhalation of the medicament by the patient. As disclosed, the audio sensor does not monitor other information such as flow rate or duration. No method is disclosed for how a sound is determined to be a delivery event. No enablement of how the audio sensor is attached on or near to the inhaler is provided.

US 2012/0265548 discloses a system and method for obtaining an indication of an incentive based on the attributes of the individual and a therapeutic component being available to the individual, transmitting the indication of the incentive to a putative provider of the therapeutic component, assigning a component of an incentive partly based on an indication of a therapeutic component administered to a portion of an individual and partly based on a profile of the individual. Incentives may include monetary, service, or other incentives. One disclosed method of acquiring an indication of a therapeutic component being administered is using auditory, visual, or other sensor data of a cover, plunger, button, or other actuator of the dispensing device in operation. Disclosed dispensing devices include an inhaler, syringe, pill dispenser, and transdermal delivery device.

US 2013/0043975 discloses a system and method for determining if a device has been ingested. The device has one or more immersion-responsive structures, mucosal material sensors, pH sensors, and auditory data distillation modules configured to detect one or more of a swallowing sound, a temperature about equal to that of a living body; a pH about equal to that of stomach acid; a pH increase indicative of travel through a small intestine and of earlier ingestion; auditory or optical indicia of ingestion; mucous or mucosa characteristic of an intestine; or an ambient pressure, electrical conductivity, or other device detectable characteristic of immersion in stool or other bodily fluids. Some disclosure of alternative routes of delivery is supplied, including inhalation and injection.

WO 2008/085607 discloses devices for the monitored storage and dispensing of medication, wherein the devices comprise a plurality of storage compartments, wherein each storage compartment has an interior space for storing at least one medication or at least one medication reminder marker; an image capturing device positionable to capture an image of the interior space of each of the plurality of storage compartments; and a communications module for electronically transmitting the image captured by the image capturing device to central monitoring station. The devices may include at least one audio, visual, or tactile means for communicating information, and may include a microphone. The device may further comprise an electronic communications component, including at least one audio, visual, or tactile means, for communicating information from a user of the dispenser to the central monitoring station.

WO 2008/091838 discloses a medicament delivery device such as an autoinjector, a pen injector, an inhaler, a transdermal delivery system or the like which includes an electronic circuit system to track the patient compliance data associated with the use of the medicament delivery device. The device includes an optional an audio output, such as recorded speech, instructing the user in the use of the medical device.

WO 2010/056712 discloses a medicament delivery device such as an autoinjector, a pen injector, an inhaler, a trans dermal delivery system which includes electronic circuit system configured to produce a recorded speech output instructions associated with, for example, stability of the dose for example of a vaccine, an instruction for using the device to be monitored, an instruction for following a regime associated with the drug, and/or a post-delivery instruction. The electronic circuit system is configured to produce a signal, such as, for example, a wireless validation signal, when the activation mechanism is actuated.

WO 2011/135353 describes a monitor for an inhalation device wherein a sound transducer is placed inside the device in the air flow path, and inhalation through the device is monitored by measuring the amplitude of the dominant frequency of the sound.

Prior art inhaler devices monitor inhalation flow rate via pressure transducer ports, microphones, or mechanical means in the inhalation flow path. These means have the problem that they can become blocked or obstructed by foreign objects from the surrounding air, exhaled matter if the patient exhales, coughs, or sneezes into or through the device, or by drug particles or dried drug. Thus, there is a need for a method of monitoring inhalation parameters in a way that does require a mechanical or pneumatic connection to the air flow path. In addition, these monitoring means and concomitant airway extension have the problem that they may affect the airflow and aerosol properties, changing them from how the device was designed, tested, and approved by a regulary agency. Thus there is a need for a method of monitoring air flow rate in a way that does not require any modifications to the device airflow path.

Prior art monitoring systems monitor the actuation of a device via a means such as electrical or mechanical that interacts with the device to be monitored's actuation and triggering system. This gives rise to the possibility that a failure or incorrect installation of the monitor can lead to a failure of the device, potentially leading to a change in the delivered dose, or no delivered dose. The monitoring system can also change the triggering characteristics of the device, for example requiring a higher triggering force or a modified triggering action relative to that which was previously tested in clinical studies and approved by a regulatory agency. Thus, there is a need for a device that monitors the triggering of a device to be monitored without a mechanical or electrical connection to the device actuator or trigger.

Prior art devices were designed to interface either mechanically, electrically, or pneumatically with a specific device in a very specific way. Thus, there needed to be a monitor specifically designed for each device. This leads to many difficulties, including the need to develop and maintain a large number of different monitoring systems, and inability to take advantage of economies of scale that would be available if there were a monitoring device that could be used in a generic way with a large number of existing drug delivery technologies, including essentially all inhalation devices.

Prior art devices have to be either factory integrated with the drug delivery system, or assembled in a way by the user that could be somewhat complex and could require partial disassembly of the device to be monitored, giving rise to the possibility of damage to or incorrect assembly of the device. Devices that are factory integrated become part of a drug product and thus can be regulated as drug products, often a significantly higher regulatory hurdle than for a medical device. Thus, there is a need for a device to be monitored monitor that can be simply adhered to a device to be monitored using, for example, an adhesive strip or pad with a release liner. Similarly, there is a need for a monitor for devices to be monitored that can be easily attached by a user or caregiver that is partially or entirely insensitive to the precise location of the monitor on the device.

Some prior art devices utilize a microphone to monitor acoustic sound pressure (with unit dimensions of force per unit area) created by a device to be monitored to detect and identify user interactions with the device to be monitored. These prior art devices are subject to signal disturbances and errors caused by environmental acoustic sound pressure coming from the environment (not from the device to be monitored). Thus, there is a need for a device monitor that is less susceptible to errors caused by environmental acoustic sound pressure.

### SUMMARY OF THE INVENTION

The invention is a monitoring system used in connection with a device to be monitored, preferably a device such as a drug delivery device which can be monitored by detecting the movement, positioning, and/or vibration of the device, such as an inhaler or a needle free injector. The monitoring system preferably includes a monitor, a means for attaching the monitor to the device to be monitored, and a display device, although some or all of these functions may be combined in one or more assemblies. The monitoring system includes a vibration sensor selected from the group consisting of an accelerometer, a vibration velocity sensor and a vibration relative motion sensor. The accelerometer detects the accelerations associated with mechanical movements and generates an electronic signal in response to the detected accelerations. The vibration velocity sensor, such as a geophone detects the velocities associated with mechanical movements and generates an electronic signal in response to detected velocities. The vibration relative motion sensor detects the change in the relative position between two locations associated with mechanical movements and generates an electronic signal in response to detected relative motions. At least one of these sensors is present and more than one may be used simultaneously.

The system further includes an electronic storage device holding stored electronic information. The stored electronic information corresponds to analysis results obtained from the analysis (including theoretical or experimental) of sensor signals associated with an operation or action. Those analysis results may be those expected from the proper operation of the device and/or may be those expected from incorrect operation of the device. The monitoring system further includes one or more software programs which identify and characterize events based on analyzing the measured electronic signals from the vibration sensor or sensors and comparing the analysis results with the stored analysis results. The program then performs a calculation, including but not limited to a best fit, measure of cross correlation, a difference between the actually detected signal and the stored expected signal analysis results, thereby determining information such as whether the device was correctly used, incorrectly used, the degree of incorrectness in the use, or another statistical measure of related to the use of the device to be monitored. Statistical measures include but are not limited to average inhalation flow rate, inhaled volume, peak flow, inhalation time, and dose delivered.

Vibration sensors, selected from accelerometers, vibration velocity sensors and vibration relative motion sensors, differ from microphones in several important ways. Vibration sensors respond to accelerations or velocities, whereas microphones respond to sound pressure waves. Sound pressure waves are compression waves, whereas vibration sensors respond to various types of oscillations, including shear, flexural, and surface oscillations. Microphones are more sensitive to ambient sound than vibration sensors. Microphones in general need to have a large area to increase response to pressure, whereas vibrations sensors, and especially accelerometers, can be very small. Microphones have moving parts, whereas accelerometers do not. For these reasons there is a significant non-obvious benefit to the use of vibration sensors over microphones for device monitoring.

A monitor and monitoring system for a device to be monitored, such as an inhaler or injection device is disclosed and described. The invention detects, interprets and thereby monitors small vibrations directly experienced or generated by a device to be monitored when it is, for example, loaded or otherwise prepared, triggered, drug is delivered, or when air is drawn through an inhaler. The vibration signals' time waveforms and/or frequency spectra are measured, analyzed and/or compared to pre-loaded analysis results related to previous or expected vibration signals' time waveforms and/or frequency spectra, and the results of this comparison may be used to identify events, for example triggering of a device to be monitored, inserting or advancing of a dosage form, shaking, setting a desired dose, delivery of a dose, opening or closing of a cap or mouthpiece, etc. In addition, identification of an event, for example a triggering event, can prompt the monitoring system to perform additional detection and/or computation, for example determining the duration of drug delivery and thus dose delivered from an autoinjector, or the inhalation flow rate through an inhaler. In addition, the amount of deviation from pre-loaded or previously measured signals' time waveforms and/or frequency spectra and/or analysis results thereof may be used to rate the quality of an event, for example determining that a dosage form strip has been fully advanced.

In one embodiment, the device to be monitored is an autoinjector similar to an insulin pen, wherein the desired dose is set by turning a dial which indicates the dose to be delivered. Turning a knob creates a series of discrete vibrations, each one of which corresponds to an increase or decrease of desired dose, for example 1 IU or 0.5 IU of insulin. By monitoring and counting the number of increases or decreases, the desired insulin dose may be monitored. Similarly, the delivery of the dose has a corresponding series of vibrations, each also associated with, for example, 1 IU or 0.5 IU of insulin. By counting these vibrations, the actual dose delivered can be monitored. By comparing the dose delivered to the desired, set dose, it can be determined if the dose was fully delivered.

Acoustic sound pressure (with units of force per unit area) measurement using a microphone can be used to monitor patient interactions with inhalation devices, including the inhalation flow rate. The current invention has numerous advantages over this technique. When acoustic sound pressure waves and mechanical vibration are created by a device, only a small fraction of the energy in the device vibration is coupled into the air as acoustic sound pressure waves. Acoustic sound pressure energy decreases as the distance from the device increases. The amplitude of detectable acoustic sound pressure decreases dramatically relative to increased distance of the microphone from the device. In addition, microphones are more sensitive than sensors which directly measure mechanical vibration to extraneous signal pickup from acoustic sound pressure coming from sources located outside of the device to be monitored. Extraneous acoustic sound pressure in some environments can have a significant adverse impact on the measurement quality (signal-to-noise ratio) of the monitor. Thus, to maximize signal quality and device monitoring accuracy, the use of a vibration sensor (such as an accelerometer, geophone or displacement measurement sensor) to directly measure mechanical vibration of the device, has significant advantages for this monitoring application, relative to the use of a microphone to measure acoustic sound pressure. For these and other reasons, there is a significant, non-obvious benefit to directly monitoring the mechanical vibrations of the device as compared to the monitoring acoustic sound pressure.

The invention is a monitoring system comprised of a vibration sensor, such as an accelerometer or a geophone, which translates vibration into electrical signals. The vibration sensor may be attached directly to the device being monitored or designed to readily attach to the device being monitored. In a preferred embodiment, the vibration sensor is attached to another body which is preferably rigid and elongated, for example a pin. The pin may be attached to the device to be monitored, but is preferably held in contact with the device to be monitored, preferably by way of a compliant element or spring incorporated in the monitoring device. The electrical signals' time waveforms and/or frequency spectra are preferably digitally acquired by a data acquisition system controlled by a software program within the monitoring system. The monitoring system includes a set of previously recorded or otherwise generated and pre-loaded previously measured vibration signals' time waveforms and/or frequency spectra and/or previously developed results developed from analysis of previously measured or calculated vibration signals' time waveforms and/or frequency spectra, which correspond to a desired or incorrect operation of a device to be monitored. The software program within the monitoring system compares an analysis of the vibration signals' time waveforms and/or frequency spectra detected by the vibration sensor with a specific pre-loaded set of corresponding analysis results. These corresponding analysis results may be from previously measured reference vibration signals, pooled results of multiple previously measured vibration signals, or generated by other means, including but not limited to theoretical means. If the comparison reveals a high degree of correlation, a small difference, or fulfills one or more other pre-specified criteria, then the vibration is identified, for example, as a successful event whereby the monitored operation of the device is judged to have been successfully carried out, or for example is identified as an incorrect operation or an incorrectly conducted operation.

Based on this identification, or other actions including but not limited to the press of a button, contact with a sensor, a change in the orientation of the device, shaking of the device etc., certain events may prompt additional computation or actions, for example turning on the monitor, acquisition and analysis of inhalation flow measurements or desired or delivered dose, or other actions, such as prompting the generation of a visual display, auditory feedback, auditory playback of a next instruction, illumination of a LED, etc. These identified events and the results of additional calculations may be provided to the user of the monitoring system and optionally others, such as their physician and/or caregiver. For example, the identification of the opening of a device to be monitored or removal of a cap may prompt an instruction, for example to load a dose, set a dosage amount, shake or prime a device, or advance a dose strip. The time, date, identification of an event, computed information such as the dose delivered, and quality of an event, such as whether an inhalation was of the correct flow rate and volume, are provided to the user, for example after the event, before a next event, and/or via a data screen. The inhalation flow rate through an inhalation device may be provided to the user in real time during the inhalation, for example via visual cues such as on the display, for example as a graph or moving bar or arrow, along with a target inhalation flow rate range, and/or via other means such as LED(s), or auditory cues. The monitoring system also may provide information to the user when the device has been incorrectly used. By way of example, with an inhalation device the program can provide visual and/or audio information indicating that the user should inhale more quickly, inhale more slowly, inhale more steadily, or inhale for a longer period of time during subsequent dosing events, or positive feedback that the inhalation was done correctly.

The monitor of the invention may be a separate device attached to the outside or be designed to be an integral set of components of a medical or other device, for example a device to be monitored such as an inhaler or injector. However, the monitor of the invention may be comprised of the vibration sensor component the display device, e.g. a smartphone, tablet or laptop computer. This vibration sensor of the display device gathers vibration data from the use of a medical or other device and translates that vibration information into an electronic signal. A software program running on the microprocessor of the monitor or on the display device analyzes and responds as described above. The display device can provide visual or auditory cues to indicate correct use or incorrect use of the device to be monitored or provide additional information including coaching the user to use the device differently such as by avoiding excessive tilting of the device to be monitored, or inhaling more quickly, inhaling longer or inhaling more slowly.

The monitor may have an integrated speaker and/or lights and/or display to provide visual or auditory cues to indicate correct use or incorrect use or provide additional information including coaching the user to use the device differently such as by avoiding excessive tilting of the device to be monitored, or inhaling more quickly, inhaling longer or inhaling more slowly. The integrated speaker and/or lights and/or display of the monitor device can also provide information regarding the operational status of the monitor, including but not limited to on/off status, battery charge status, data pairing status and data communication status. Preferably some or all of these functions are included in the display device.

A medical device in the form of a drug delivery device having a monitoring system connected thereto is disclosed. The medical device may be any type of medical device which undergoes changes in the position, vibration, or movement during use and includes devices used for aerosolized drug delivery and the injection of drugs including insulin. The monitoring system is comprised of a monitor comprising a sensor which generates an electronic signal. The sensor may be an accelerometer, a vibration velocity sensor, or a relative motion sensor. The sensor transmits the electronic signal to a processor for analysis, for example comparing the signal to a standard stored in the monitor or stored elsewhere. The standard corresponds to a known activity of the device which may be proper use of the device or improper use of the device. On making the comparison, information is then transmitted indicating that the device has been properly operated or improperly operated and may provide additional information on the use of the device including whether the drug was properly delivered and/or should be re-administered.

In one embodiment, the device to be monitored, preferably a dry powder inhaler, is monitored for tilt whenever the powder medicament is exposed and ready for inhalation. If the measured tilt exceeds a predetermined angle, the user is presented with audio feedback, preferably in the form of a rapidly repeating tone or beep. As the tilt increases, one or both of the audio frequency (pitch) and beep repetition rate increases. The tone may be generated either by the monitor or by the display device. In the embodiment where the tone is generated by the display device, the tone is preferably generated even if the display device is not being held or observed by the user, for example the display device is a smart phone in the pocket or purse of the user, and the monitoring application is running but not in the foreground, i.e. it would not be visible on the screen even if the screen were on. Many inhalers, for example the Diskus device (GSK), mechanically block the airway if a dose is not ready for inhalation so that the patient doesn't inhale at the incorrect time. In the Diskus device, the airway block is removed by the same mechanism that advances the dose strip and removes the cover from the dose blister. Thus, after inhalation, the configuration of the device is that the airway is open and no dose is present for delivery. In a preferred embodiment of the invention when used with this type of inhaler, if the user is looking at the display device following an inhalation delivery event, they are presented with the option of entering a “patient training mode”. in the patient training mode, the user is taken through the steps of the inhalation (but not the steps of preparing the dose for inhalation) while being given real time feedback and guidance on inhalation rate and volume. Following the training inhalation, they are given feedback on the quality of the inhalation and suggestions for improvement, and given the option of doing another training inhalation. If the patient has previously performed inhalations that were not optimal, for example too fast, slow, shallow, or variable, they are preferably presented with a suggestion, either by the display device directly, or via other means including but not limited to email or text message, to use the patient training mode to improve the quality of their inhalation maneuver.

The monitor of the invention comprises a vibration sensor, an electronic storage device which holds stored electronic information corresponding to a recorded or otherwise generated vibration signal's time waveforms and/or frequency spectra related to vibration generated by a desired or incorrect operation of a device to be monitored, and a program which compares an analysis of the vibration signals' time waveforms and/or frequency spectra detected by the vibration sensor with a specific pre-loaded set of corresponding analysis results device to be monitored. The vibration sensor may be an accelerometer or similar device such as, but not limited to, a geophone or other vibration velocity sensor or a vibration relative motion sensor (of optical, capacitive, resistive, or inductive type) which directly detects the physical motion of a selected component of the device itself (actual mechanical movement/acceleration of a solid). More specifically, it is not an acoustic sensor detecting air pressure waves, which travel away from the device or are in the air that is inside the device, but is a sensor of mechanical movement or acceleration of a selected solid device component. The vibration sensor may be a DC (direct coupled) accelerometer or other DC sensor capable of measuring tilt as well as higher frequency vibrations.

The monitoring system may include a display device with a screen which displays information relating to the analysis result from the monitored vibration signals. The program may evaluate the calculated difference and identify correct operation of the device to be monitored when the calculated difference is less than a predetermined amount or by means of another algorithm. The program may generate operations and result in providing feedback and/or guidance and/or instructions, calculating an inhaled flow rate, calculating an inhaled volume, displaying an inhalation flow rate, calculating a delivery dose or other operation generally used in connection with devices to be monitored.

The invention includes one or more software programs which can be loaded onto a computing device such as a smartphone, smartwatch, glasses, tablet, laptop or desktop computer or the like. The software includes a program operation for translating a vibration signal's time waveforms and/or frequency spectra obtained from a direct, mechanical vibration sensor into a defined pattern. The program utilizes a preloaded set of analysis results and/or standard patterns obtained from analysis of previously measured vibrations generated from the proper use of a given device to be monitored such as a drug delivery device. The program includes a means for comparing the standard pattern or patterns with a defined pattern obtained from analysis of the vibration signal's time waveforms and/or frequency spectra from the vibration sensor. The program may also includes an operation for computing a measure of the difference between the defined pattern and the standard pattern as well as a means for generating a display of a visual image based on the results of the analysis or the correlation or differential. The program may include operations which generate information for the user of the device to be monitored such as a drug delivery device which is intended to aid the user of the device to be monitored in providing for more optimal and consistent usage, such drug delivery and treatment of a patient.

The device to be monitored can be any device that makes measurable mechanical vibrations, for example a drug delivery device when a dose is loaded, advanced, prepared, shaken, inhaled, injected, or ingested.

In one embodiment, the device to be monitored is a medication inhaler. The monitor can sense actions including but not limited to motion of the inhaler including picking it up or shaking it, priming, removal of a cap or cover, setting of a breath actuation mechanism, insertion of a unit dose dosage form or multidose reservoir, advancement of a dose strip, metering of a dose from a multidose reservoir, exhalation of the patient prior to delivery, triggering of the inhaler, inhalation rate, duration, and/or volume through the inhaler, or exhalation of the patient after the delivery, for example after a breath hold. The monitoring system can present the patient with information including but not limited to when to dose, reminders to dose, how many doses or what dosage to deliver, which dosage form to use, reminders and training as the proper use of the device including shaking, exhalation prior to use, proper inhalation flow rate and inhalation volume, actual inhalation flow rate and inhaled volume, breath hold reminders and countdowns, and summary information from current and previous drug delivery events. The monitoring system can also incorporate information related to number of remaining doses, expiration due to time since the dosage form was removed from its primary packaging, and/or expiration due to shelf life. The monitoring system may be combined with an additional device such as a pulmonary function meter, and using data from the meter, suggest actions including but not limited to dosing, skipping a dose, and/or dose amount.

In another embodiment, the device to be monitored is a parenteral delivery device, including but not limited to autoinjectors, prefilled injectors, needle-free injectors, pumps including patch pumps, bolus pumps, wearable pumps, pole mount pumps, and the like. The monitor can sense and detect actions including but not limited to motion of the injector including picking it up or shaking it, insertion of a unit dose dosage form or multidose reservoir, advancement of a dose strip, metering of a dose from a multidose reservoir, triggering of the injector, removal of a cap or cover, setting of a dose, closing and/or locking of a controlled substance drug enclosure, and/or duration of the delivery. Duration of delivery can be monitored, for example, by listening to the amount of time the delivery takes, e.g. the vibration of a motor, the vibration of drug flowing through the system, multiple vibrations associated with delivery of preset amounts of drug, for example 1 or 0.5 IU of insulin, and/or the duration of time from a triggering event to an end event such as a piston hitting a stop. The monitoring system can present the patient with information including but not limited to when to dose, reminders to dose, how many doses or what dosage to deliver, which dosage form to use, reminders and training as the proper use of the device including shaking, cleaning of injection site, injection duration reminders and/or countdowns that remind a patient how long to keep an injection device in place, and summary information from current and previous drug delivery events. The monitoring system may be combined with an additional device such as a blood glucose meter, and using data from the meter, suggest actions including but not limited to dosing, skipping a dose, and/or dose amount.

In another embodiment, the device to be monitored is a container for dosage forms including but not limited to pills, capsules, films, troches, lozenges, pastilles, suppositories, powders, liquids, solutions, suspensions, or unit dose or multidose drug containers designed for use in another drug delivery system including but not limited to inhalers, injectors, pumps, transdermal, nasal systems, sprays including but not limited to sprays for nasal, ocular, dermal, or buccal administration. The monitor can sense actions including but not limited to picking up the container, opening the container, removing the dosage from the container, which well of a multi-well container was opened, and the like. The monitoring system can present the patient with information including but not limited to when to dose, reminders to dose, how many doses or what dosage to deliver, which dosage form to use, which medication to deliver, reminders and training as to the proper use of the dosage form including but not limited to shaking, diluting, sucking, swallowing, sprinkling, or dissolving, and summary information from current and previous drug delivery events.

In one preferred embodiment, the drug is a controlled, commonly abused substance, or dangerous substance with high overdose potential, including but not limited to an opioid or other pain medication, alcoholic beverage or other alcohol containing substance, barbiturate, benzodiazepine (particularly alprazolam, lorazepam, and clonazepam), cocaine, or methaqualone. The monitor can sense use of the substance for example by sensing the opening of a container or use of a device to be monitored, and how much is used. The monitoring system can supply a patient or skilled or unskilled caregiver with information related to time since last use, amount used, allowable amount to use, time to next allowed dose, etc. If the usage exceeds allowed amounts, the monitoring system can give a notification to the user or caregiver, or transmit a notification to a person or persons including but not limited to family members, friends, nurses, physicians, poison control centers, emergency medical personnel, or law enforcement authorities. This notification can be sent by any means, such as voice messaging, email, text messaging, ‘tweeting’, and/or posting to a web site. It will be obvious to one skilled in the art that future means of sending notifications will be developed that can be used by the device.

In another embodiment, the device to be monitored is used to deliver a drug to multiple different patients, for example for mass vaccination campaigns, bioterror response, and the like. The monitoring system can monitor the usage of the drug delivery system, give training and feedback to the operator, monitor for correct device operations, measure frequency of dosing, number of doses, location of dosing events, etc.

The monitoring system can also be used with any other device that requires that use or other activity is monitored, must be used in a prescribed and/or controlled variable way, and makes at least one measurable vibration signal.

Many drug delivery devices deliver drug from a reservoir, and the amount of drug is controlled by the duration of the delivery. Examples include but are not limited to injectors, infusion systems, pumps, inhalers, nasal delivery systems, transdermal systems, and the like. In one embodiment of the current invention, the monitoring system captures the duration of a vibration created by the delivery and/or the time duration between or the count of two or more vibrations characteristic of the dose delivered, and based on a previous laboratory evaluation of the drug delivery device and optionally the concentration of the formulation and/or the number of doses remaining in a reservoir, calculates and stores a dose delivered. In a preferred embodiment, the device to be monitored is an autoinjector. In a particularly preferred embodiment, the autoinjector comprises insulin or an insulin analog. Preferably, the monitoring system prompts the patient to continue the injection, for example by keeping a needle, catheter, or the like inserted or by keeping the autoinjector pressed against the injection site, and to continue actuating the delivery, for example by pressing on a plunger, button, or other means, until the monitoring system determines that the delivery is complete or a predetermined time has elapsed. In another embodiment, the device to be monitored is a bolus injector, and the monitoring system prompts the patient to remove the bolus injector when the injection or infusion is complete. In yet another embodiment, the device to be monitored is a pump, and the monitoring system displays information related to infusion rates, bolussing events, occlusions, device failures, dose remaining, and reminders to change infusion sets, refill, recharge, and/or change batteries.

In one embodiment, the device to be monitored is a system, for example an inhaler, that generates an aerosol from one or more orifices or nozzles. These systems can be vulnerable to blockage or constriction of nozzles due to particulates or dried dissolved components such a drug. In general, as this blockage progresses, the nature of the delivery changes, for example the duration of a delivery event increases. The monitor of the current invention can monitor this change, and when it exceeds a prespecified threshold, such as a delivery duration, the user can be prompted to replace the device to be monitored or a component thereof such as a nozzle array or orifice plate.

In one embodiment, during a training event the monitor acquires a sample and stores the vibration signal's time waveforms and/or frequency spectra and/or analysis results thereof, which become reference results. In the embodiment wherein the training event is conducted more than once, the measured vibration signals are analyzed and information related to the average and/or variation or other attributes of the signals' time waveforms and/or frequency spectra may also be stored. These reference results are then compared with the corresponding results of subsequently acquired signals' time waveforms and/or frequency spectra, and a goodness of fit algorithm or other method is used to determine if an event has occurred. In one embodiment, the reference results are stored on a separate display device. Because this may require that the display device be available, be powered on, and be running the software during a dosing event, in a preferred embodiment the signal and reference time waveforms and/or frequency spectra and/or analysis results thereof are stored on the monitor, and the signal's time waveforms, frequency spectra, and/or results of computations can be transmitted the display device at a later time. The goodness of fit determination can be conducted by the display device, and preferably is, in the embodiment where the reference signals' time waveforms and/or frequency spectra and/or analysis results thereof are only stored on the display device. In a preferred embodiment, the signal's time waveforms and/or frequency spectra and/or analysis results thereof are stored on the monitor, and a goodness of fit determination is made by the monitor as a criterion for storage, and possibly later transmittal to the display device. In a preferred embodiment, the goodness of fit assessment done by the monitor is preliminary, based on a few parameters such as amplitude, duration, etc, and this assessment is used to determine if the signal's time waveforms and/or frequency spectra and/or analysis results thereof should be stored. Subsequently, when connected, preferably wirelessly, to the display device, data are downloaded, and a final goodness of fit determination and determination of other parameters such as, in the embodiment where the device to be monitored is an inhaler, inhalation flow rate, duration, etc. are performed. In some embodiments, only analysis results of the event are stored on the monitor and/or display. In a preferred embodiment, the full signal's time waveforms and/or frequency spectra are stored by the display device, allowing for future changes to the software and/or display configurations. In another embodiment, The monitor simply stores all data acquired for a predetermined amount of time after an event, such as activation of a turn on switch, movement of the monitor, or recognition of a an event such as opening or otherwise preparing the device to be monitored for use. Recognition of the event can be done via measured vibration, or by another means including but not limited to mechanical, electrical, audio, or optical. The stored data is transmitted to the display device immediately or at a later time when the monitor has a data connection to the display device, and the analysis of the vibration data is done by software on the display device. In yet another embodiment, the monitor simply acquires vibration data at all times the monitor is attached to the device to be monitored, and transmits the data and clears it memory when a data connection to the display device exists.

In one embodiment, the location of the monitor is completely at the discretion of the user, patient or caregiver, and can be modified based on the user's preferred method of using the device to be monitored. In a preferred embodiment the user is given a suggested or required location for attachment of the monitor based on the specific device to be monitored. Depending on the ability of the patient to accurately locate the device, and the sensitivity of the algorithm for identifying an event to the amplitude or amplitudes of the event, the device may or may not require additional calibration.

The monitor may be attached to the device to be monitored, and optionally calibrated, in the factory where the device to be monitored is fabricated. Alternatively, monitors may be attached in a controlled, batch process and optionally calibrated by a third party, for example a pharmacy, hospital, HMO facility, doctor's office, etc. In a preferred embodiment, the monitors are attached individually by the user, patient or caregiver, and optionally calibrated by performing the desired event, such as triggering, inhaling, etc, in a “monitor training mode”.

Any means of attachment can be used, such as fasteners, adhesives, elastic bands, etc. In a preferred embodiment, the attachment is achieved using an adhesive strip or pad, which is supplied with the adhesive covered by a removable release liner. In one embodiment, the adhesive is supplied with a cleaning means, such as a solvent or other cleaning agent infused in a cotton swab or cloth. In a preferred embodiment, the nature of the adhesive, size and shape of the monitor, and/or the size of the carrier or strip are such that no surface preparation is required. In one embodiment, the monitor is supplied with the adhesive already attached and ready for use after removal of a release liner. This is preferred for the embodiment wherein the monitor is non-releas ably attached to the device to be monitored. In another preferred embodiment, the carrier is in attached to another component to form a carrier for the monitor, and the monitor is removably attached to the carrier. In a preferred embodiment, the carrier contains a through hole, and the monitor comprises a mating, feature which contains or is attached to the vibration sensor. The monitor's raised feature is inserted into the through hole as the monitor is attached to the carrier, for example by a friction fit, screw thread, bayonet fitting a detent which supplies positive feedback in the form of a click to the user that the monitor is properly inserted. In one embodiment, when the carrier is attached to the device to be monitored and the monitor is attached to the carrier, the vibration sensor is in contact with the device to be monitored, maximizing the vibration level and shielding it from ambient acoustic sound pressure. In another embodiment, the vibration sensor is attached to an elongated, rigid component which extends out of the monitor and through the hole in the carrier, contacting the device to be monitored. The carrier comprises an adhesive and a release liner, and is non-releasably attached to the device to be monitored. The carrier may be supplied in a single form that is applicable to all expected uses of the monitor, and for example may be flexible, for attachment to rounded or otherwise contoured device surfaces, for example insulin pens. In another embodiment, the carrier may be specific to a device or set of devices, contoured to mate to the surface of the device or devices, and may optionally include a footprint or other fiducial features to aid in proper placement on the device. In one particularly preferred embodiment, the device is a “Diskus” device, such as that used by the GSK “Advair” drug product. The Diskus has a flat circle approximately 30 mm in diameter, with a raised feature along its circumference, on both the top and the bottom of the device. The carrier may include a portion of its edge which is of the same radius as the raised feature, and can interface with the raised feature. The carrier in this embodiment would have a through hole which may be centered on the radius of curvature of the radiused portion of its edge. In this way, the vibration sensor would always be in the same spot at the center of the Diskus flat circle, independent of where on the circumference of the flat circle the edge of the carrier is placed.

In another embodiment, the monitor may be incorporated into the device to be monitored when the device is manufactured. In this embodiment, the vibration sensor may be preloaded against a suitable component of the device to be monitored using a compliant element, either directly or through an additional rigid component to which the vibration sensor is attached, in a way similar to that described above. In a preferred embodiment, the vibration sensor is attached directly to a suitable component of the device to be monitored, for example through the use of a suitable adhesive. In a still more preferred embodiment, the vibration sensor is a component on a printed circuit board, and the board is sufficiently rigid, and rigidly attached to the device to be monitored such that the vibration sensor responds to the device vibrations that are transmitted through the circuit board. The circuit board may be dedicated to the monitor, or preferably may have other functions such as motor controllers, display drivers, additional sensing functionality, and the like.

The monitor may be supplied with one or more carriers when packaged for sale. A multiplicity of carriers, possibly different versions depending on the device to be monitored, are preferably also offered for separate sale.

The monitor preferably includes a means for transmitting acquired data to another device for display, analysis, and/or subsequent re-transmission. The transmission to the display device can be by a wired means including but not limited to USB or firewire, but is preferably by a wireless means such as wifi or Bluetooth, preferably Low Energy Bluetooth. It will be obvious to one skilled in the art that future wired and wireless communication protocols and systems will be developed and can be used by the invention. The display device may be a dedicated system supplied with the monitor, but in a preferred embodiment is a device the user already owns and/or would be useful for other activities, such as a smartphone or tablet. Preferred display devices include but are not limited to mp3 players, smartphones including but not limited to Android phones, iPhones, Blackberry devices, or Microsoft phones, smart watches and other wearable devices, eyeglasses capable of displaying information such as Google glass, tablet, notebook, and desktop computers, automobiles, televisions, and television connected peripherals such as DVD players, Blue-ray players or streamers. It should be noted that electronics technology is rapidly evolving, and it will be obvious to one skilled in the art that related but new display and analysis technologies will be available in the future, and may be used with the current invention.

Although a vibration sensor is less susceptible to errors and noise pickup caused by the external acoustic sound pressure in the environment, it still will have some sensitivity to disturbance from environmental acoustic sound pressure. Thus, preferably the monitor and/or carrier contain features that shield the vibration sensor from ambient acoustic sound pressure. In a preferred embodiment, the monitor includes noise cancelling technology, which may comprise a microphone which measures the ambient acoustic sound pressure environment, and functionality for subtracting a correction signal's time waveforms and/or frequency spectra and/or analysis results thereof obtained by processing the acoustic sound pressure signal's time waveforms and/or frequency spectra and/or analysis results thereof from the vibration signal's time waveforms and/or frequency spectra and/or analysis results thereof acquired by the vibration sensor. The correction signal's time waveforms and/or frequency spectra and/or analysis results thereof for the subtraction may be simply proportional to the time waveforms and/or frequency spectra and/or analysis results thereof obtained directly from the acoustic sound pressure signal, or may be multiple constants or otherwise contoured, for example frequency filtered. The function used for creation of the correction signal's time waveforms and/or frequency spectra and/or analysis results thereof may be calibrated prior to a delivery event. In one embodiment, the patient is instructed or prompted to hold in position (if required) immediately prior to delivery for a predetermined amount of time, for example with a needle inserted or an inhaler in the mouth, and the ambient acoustic sound pressure measured by the microphone, and the associated vibration measured by the vibration sensor are monitored. In this way the noise cancelling can be calibrated in a way optimized for the acoustic environment in the exact configuration that the delivery will take place.

In the embodiments with or without noise cancelling, the monitoring system may monitor the ambient acoustic sound pressure and/or vibration levels, and take an action such as give an instruction to the user in the event that the ambient levels exceed prespecified values in one or more frequency ranges. For example, the user may be instructed to move to a quieter location, or the data acquired may be flagged as potentially corrupted.

The acquired data can be used in many ways. In one embodiment, analyzed data may be displayed to the user in real time, while he or she is using the device to be monitored, for example delivering drug, as a method of training and feedback. For example, in the embodiment where the device to be monitored is an inhaler, the patient can be prompted to inhale more slowly or more rapidly, continue inhaling, or trigger the device. In another embodiment, the patient is given feedback on the quality of the delivery maneuver after the delivery event, so he or she can, for example, improve the maneuver at the next dosing event, or repeat the delivery if it is determined that an insufficient dose was received. The data can also be displayed in tabular form, displaying all of the events available, all of the events in a requested or predetermined interval, all of the events with a particular device to be monitored (when the display device is used with more than one monitor), etc. These events can be displayed in many ways, such as in tabular form, graphical form, and/or in a way that highlights incorrectly conducted delivery events. In an additional embodiment, the patient or caregiver enters additional data, such as the information related to, for example, the time, date, and severity of a medical event, such as an asthma exacerbation. Such data can also be entered automatically, for example from an electronic medical record. In addition, the data from many patients can be combined and used to determine how well a population uses a device to be monitored, optionally combined with location data such as GPS. These data can be used, for example, in clinical trials, post marketing commitments, or scientific studies, for example to determine which devices are the easiest to use properly, how users such as patients use or misuse devices. In the embodiment where the device to be monitored is a drug delivery device, these data can also be used to determine which delivery profiles and compliance result in the best clinical outcomes, and the instructions, training, and/or feedback can be modified accordingly to improve patient and patient population clinical outcomes and reduce direct and indirect costs associated with the condition.

Software for the display system may be supplied with the monitor, but is preferably downloaded by the user from a web site, application store, or the like. Preferably the application allows the user to select the device to be monitored, and instructions, calibration, images etc. are downloaded that are specific to that device. The software may also allow an option that is generic to any drug delivery or other type of device. In this embodiment, the display unit is used to put the monitoring system in a “monitor training mode”, and the desired event, for example device actuation, is conducted, preferably 1 time, but possibly 2, 3, or more times, while the vibration signal's time waveforms and/or frequency spectra and/or analysis results thereof are acquired by the monitoring system. This signal's time waveforms and/or frequency spectra and/or analysis results thereof, or average of multiple signals' time waveforms and/or frequency spectra and/or analysis results thereof, becomes a reference, and is used to identify subsequent events. While the invention is preferably directed toward drug delivery devices, it can be seen that such a generic system could be used for other applications, such as the ringing of a doorbell or phone, the opening of a refrigerator, medicine cabinet or the like, or any of a number of other applications wherein a list of event times and dates would be useful. The software would preferably comprise locked versions, for example for class II devices and others that require regulatory approval of the software. Open source versions may also be made available for development of optimized applications for lower risk medical and non-medical devices.

In one embodiment, the monitoring system acquires and analyzes the reference vibration time waveform and/or one or more frequency spectra during an initial or training event, and the vibration time waveform and/or one or more frequency spectra and/or analysis results thereof is subsequently stored. In the embodiment wherein the training event is conducted more than once, information relating to the variation in the reference time waveform and/or one or more frequency spectra and/or analysis results thereof may also be stored. This reference time waveform and/or frequency spectra and/or analysis results thereof are then compared with subsequently acquired signals' time waveforms and/or frequency spectra and/or analysis results thereof, and a comparison algorithm is used to determine if an event has occurred. In one embodiment, the vibration information is sent directly to the display device as it is acquired, where it is analyzed, and stored. Because this would require that the display device be available, powered on, and running the software, in a preferred embodiment the acquired signals' time waveforms and/or frequency spectra and/or analysis results thereof are stored on the monitor, and are transmitted to the display device at a later time, when the display device and monitor have a data connection. The goodness of fit or other determination can be conducted by the display device, and preferably is in the embodiment where vibration information is sent directly to the display device. In a preferred embodiment, the signal's time waveforms and/or frequency spectra or predetermined characteristics of the signal's time waveforms and/or frequency spectra of an identified event are stored on the monitor, and a goodness of fit determination is made by the monitor as a criterion for storage. In one embodiment, the reference time waveform(s) and/or frequency spectra and/or analysis results thereof are stored on the monitor, and the comparison of an acquired signal's time waveform and/or frequency spectrum and/or analysis results thereof to the reference time waveform and/or frequency spectrum is conducted by the monitor to identify and store events. In a preferred embodiment, the identification of an event done by the monitor is preliminary, based on a few parameters such as amplitude, duration, etc, and this assessment is used to determine if the signal's time waveform and/or one or more frequency spectra should be stored. Subsequently, when a data connection exists to the display device, data are downloaded, and final goodness of fit and/or other determinations, and final determination of other parameters such as inhalation flow rate, dose delivered, duration, etc, are performed. In some embodiments, only measured parameters of the event are stored on the monitor and/or display. In a preferred embodiment, the signals' complete time waveforms and/or frequency spectra are stored by the display device, allowing future reanalysis and display in the case of, for example, future changes to the software and/or display configurations.

In a preferred embodiment, in addition to identifying events, the monitoring system is capable of making multiple rapid sequential measurements of direct mechanical vibration and using those measurements to create a time series characterization of a time varying event. Preferably this event is an inhalation through a pulmonary drug delivery device, and the monitoring system calculates the inhalation flow rate through the device based on detected mechanical vibrations. The calculation may use one or more characteristics of the vibration, including but limited to single measurements, averages, or filtered measurements of amplitude, RMS, or the amplitude or RMS amount of vibration of the time waveform or in one or more frequency bands of the vibration frequency spectrum. The frequency bands may be defined by hardware filters, software filters, Fourier transforms, and the like. The characterization may also involve computations using multiple characteristics of the vibration, such as sums, quadrature sums, ratios, or more complex computations. To create a time series, the computation may be conducted on multiple time slices of the vibration of an event. For example, the computation may be conducted on time waveforms or spectra sampled for one second or less, preferably 0.5 second or less, more preferably 0.1 second or less. In a particularly preferred embodiment, the event is a patient inhalation through an inhalation device, the inhalation flow rate is calculated based on analyzing sequential, and possibly overlapping time waveforms of about 0.1 second duration, the inhalation flow rate is calculated by a method selected from the total rms vibration in the time waveform, the vibration in a frequency band around the actual or expected peak of the spectrum, the vibration in a frequency band previously determined to have the most power to discriminate the flow rate, the vibration in a band that includes 1 kHz, the vibration in a band that includes 700 Hz, the vibration in a band from about 4 to about 16 kHz, the ratio of rms vibration in two frequency bands, the ratio of rms vibration in a band from about 4 to about 16 kHz to the vibration in a band that includes 700 Hz, and combinations thereof, and feedback based on the calculated inhalation flow rate is presented to the patient to train or prompt an inhalation flow rate within a desired range.

Many methods of comparing a measured signal's time waveform and/or frequency spectrum to reference time waveforms and/or frequency spectra can be carried out, including but not limited to calculating a cross correlation of a signal and reference time waveforms and/or frequency spectra or calculating a cross correlation of the amplitudes of a signal and reference time waveform and/or frequency spectrum, and identifying an event based on the height of the cross correlation, calculating a residual sum of squares or other measure of the difference between the sample and reference time waveforms and/or frequency spectra or amplitudes thereof, and identifying the sample signal as an event if the measure of the cross correlation is greater than and/or the difference is below a threshold value. Alternatively, the identification of an event may be simply based on comparing the amplitude of the signal and reference time waveforms and/or spectra at certain specified points, or other analysis and/or pattern recognition analysis techniques and methodologies, including but not limited to techniques currently used in voice recognition.

In one embodiment, the software is specifically designed for a specific device, formulation, and disease state. This can be done by having a different version of the software available for each combination of specific device, formulation, and disease state. In a preferred embodiment, there are one or at most a few versions of the software available, and the device, formulation, and/or disease state are entered by the user using the display device. Based on the selected combination, the display device can select and/or download items such as the data to display, sample time waveforms and/or frequency spectra and/or analysis results thereof, goodness of fit algorithms, parameters to calculate, acceptance ranges, and optionally where to share the data. For privacy, the user can be prompted to “opt in” to data sharing with one or more other selected person's or entities. The display device may also upload certain information related to the signal's time waveforms and/or frequency spectral shape, expected amplitude and duration, fit parameters, etc. to the monitor. Optionally, the patient or caregiver can customize what data are acquired and/or displayed, and how the data are displayed, and ranges for highlighting a given datum, for example in another color such as red.

Optionally, the patient or caregiver may be prompted to enter personal information. This information may include but is not limited to height, weight, body mass index, sex, race, age, disease state, disease severity, and/or pulmonary function parameters including but not limited to vital capacity, peak expiratory flow, and FEV1. Using information selected from these input parameters and optional data from a calibration or training event, relative values measured by the monitoring system may be displayed as absolute values. For example, vital capacity may be known and entered into the device. The patient may then be prompted, during a training event, to exhale fully, and then inhale as deeply as possible through the inhaler, essentially performing a vital capacity maneuver. Based on the duration of the inhalation, and the previously measured vital capacity, an average inhalation flow rate can be computed. Based on this average flow rate, the signal's time waveform and/or one or more frequency spectra of the vibration during the inhalation, and optionally a physical model that may include corrections for such things as device flow resistance, a calibration of the flow rate vs. analysis results from measured signal time waveform and/or frequency spectrum can be established. Similarly, the patient may be prompted to enter peak expiratory flow or FEV1, and inhale or exhale through the device as rapidly as possible, to establish the calibration. Preferably, if parameters such as pulmonary function parameters are not known at the time of calibration, the device will still operate. In one embodiment, the device uses model predictions of pulmonary function parameters, based on inputted data selected from a list including but not limited to height, weight, body mass index, age, sex, race, disease state and severity. In another embodiment, flow rates and inhaled volumes are displayed as a percentage of the maximum values specific to the patient as determined during a training maneuver. In a preferred embodiment, the data are stored in such a way that actual flow parameters may be computed if pulmonary function parameters are entered at a later date, for example after a visit to a pulmonologist or asthma specialist.

In a preferred embodiment, the inhalation or other flow rate calibration is conducted in a way that is independent of the amplitude of the vibration measured during an inhalation event. For example, increasing levels of turbulence may be expected to occur at higher flow rates, which may lead to changes in the measured vibration spectrum of the device, for example higher amplitudes in some frequency bands, such as higher frequency bands, relative to other, for example lower, frequencies. Thus by comparing the ratio of amplitudes in two or more frequency bands, or other for example more complex computations, the flow rate through the inhaler may be determined based on previous laboratory measurements of the specific inhaler being used, in a way independent of the amplitude of the vibration, for example due to variations in placement of the monitor. The spectral parameters may be determined by several methods, including but not limited to band pass filters or Fourier transforms. Subsequently, measurements of the flow rate may be based on spectral measurements, but are preferably based on a calibration of the signal's time waveforms and/or frequency spectra amplitude performed via the above analysis.

In one embodiment, event signals' time waveforms and/or frequency spectra are analyzed using parameters determined during the initial calibration or training, or parameters that are downloaded. In a preferred embodiment, the parameters are recalculated after every event that satisfies the goodness of fit criteria, based on a weighted or unweighted average of the analysis results from a predetermined number of previous events. In this way, if the vibration signal's time waveform or frequency spectrum, for example of the device triggering, change over time due to any reason, including but not limited to wear of mechanical components including but not limited to triggering, actuation, and detente mechanisms, changes in the vibration properties of the device due to, for example, drug build up in the airway or changes in formulation volume contained in a drug reservoir, and/or changes over time in the location of the sensor, the reference time waveform and/or frequency spectrum will evolve accordingly.

In a preferred embodiment, delivery events are stored with a time and date stamp. In the embodiment where the display device must be connected at the time of the event, the time and date stamp can be generated by the display device using its internal clock. In a preferred embodiment where the monitor need not be connected to the display device, the time and date stamp may be generated by the monitor and stored with other data related to the event. In another embodiment, the monitor may have a simple counter such as a seconds counter or an oscillator and counter. The display device when first connected can then determine the time and date corresponding to a given count. In addition, when the display is connected multiple times, the display device can correct for inaccuracies of the monitor counter.

For portability and ease of use, the monitor is preferably battery powered. The monitor may have replaceable or rechargeable batteries or cells. In a preferred embodiment, the batteries have sufficient lifetime as compared with the expected life of the monitor that they need be neither charged nor replaced. In another preferred embodiment, the batteries are integrated with the carrier or adhesive strip, and are changed when the carrier is changed without requiring additional action on the part of the user. The monitor may comprise an additional power source, such as a second battery or a capacitive storage component, or non-volatile memory, to maintain stored events during a battery change or complete battery discharge. It will be obvious to one skilled in the art that novel power sources may be developed in the future that could be used to power the device.

Adhesive may be used to attach the monitor fixedly to a durable device to be monitored. Depending on the lifetime of the device to be monitored, the batteries may need to be replaceable or rechargeable. In a preferred embodiment, the adhesive is used to affix the monitor to a multidose disposable device, such a dry powder inhaler or a metered dose inhaler, or to a multidose disposable component of a durable device, such as a drug cartridge or battery pack. In this embodiment, the monitor may be detachable from the adhesive strip. The monitor may come supplied with multiple adhesive strips, and a new strip may be used to attach the monitor to a new device or drug cartridge. Optionally, the adhesive strip may be integrated with a battery, simplifying use of the monitor by combining the acts of replacing the adhesive strip and replacing the battery. In a particularly preferred embodiment, the monitor is attached essentially unremoveably to a device to be monitored with a limited lifetime, for example a multidose disposable drug delivery device or drug cartridge, the batteries of the monitor do not require replacement or recharging for the lifetime of the disposable device to be monitored or other component, and the monitor is disposed of at the time of disposal of (preferably with) the device or component.

The monitor includes a vibration sensing component which comprises a vibration sensor (such as an accelerometer, vibration velocity sensor or vibration relative motion sensor) for sensing the vibration of the device being monitored. While in general the vibration sensing component can be located anywhere in the monitor, in a preferred embodiment, the vibration sensing component is associated with the adhesive component, and when the monitor is attached to the device to be monitored, the vibration sensing component is held in contact to that surface by the adhesive. For example, the adhesive may be an adhesive strip or pad containing a hole through which extends the vibration sensing component. In this way, the sensor can be made more sensitive to vibrations made by the device to be monitored, and less likely to get a false positive or other interference from external vibrations. The vibration sensor may be in rigid contact with the device to be monitored. Preferably the vibration sensing component comprises the vibration sensor in rigid contact with another, preferably metal, rigid component that is in rigid contact with the device to be monitored, or in turn in contact with a third or additional rigid components the last of which is in rigid contact with the device to be monitored. In one embodiment, the vibration sensor is in rigid contact with a component such as a circuit board, and the circuit board is in rigid contact with a case, which is in rigid contact with the device to be monitored. In another embodiment, the circuit board comprises the device to be monitored. In a particularly preferred embodiment, the monitor is attached to a carrier which is adhered to the device to be monitored, the carrier contains a through hole, and the monitor's vibration sensor does not directly have contact with the device but instead the vibration sensing component comprises an additional rigid component attached to the vibration sensor and the additional rigid component is preloaded into contact with the device to be monitored. The vibration sensing component preferably comprises an elongated portion such as a spike or rigid extension pin that extends outside of the monitor.

The vibration sensing component is kept in contact with the device to be monitored by means of a preload mechanism, preferably a compliant element such as a compressed gas or mechanical or preload spring, a polymer element such as rubber, or more preferably by a foam, for example a polyurethane or a silicone foam, most preferably an extra-soft silicone foam or ultra-soft silicone foam with low stress relaxation, high compressibility and resistance to UV, ozone, and temperatures extremes. Preferably, prior to installation of the monitor onto the device to be monitored, the vibration sensing component is held against a stop by the preload mechanism, preferably by a force of about 0.004N to about 0.9N, more preferably by a force of about 0.04N to about 0.4N, most preferably by a force of about 0.1N to about 0.2N. When the monitor is installed on the device to be monitored, the preload is preferably selected so that contact of the vibration sensing component with the device to be monitored is maintained over the range of vibrations expected during the use of the device to be monitored, preferably up to about 25 g, more preferably up to 40 g, most preferably up to 50 g or higher. Preferably the vibration sensing component weighs less than about 10 gms, more preferably less than about 5 gms, still more preferably less than about 2 gms, most preferably less than 1 gm. Preferably the preload mechanism holds the vibration sensing component into contact with the device to be monitored with a force of at least the mass of the vibration sensing component times the peak expected acceleration, preferably with a force of about 0.05 N to about 5 N, more preferably with a force of about 0.1 N to about 2 N, still more preferably with a force of about 0.2 N to about 1 N, most preferably with a force of about 0.3 N to about 0.8 N. Preferably the preload mechanism has a spring constant of about 100 N/m to about 2000 N/m, more preferably about 300 N/m to about 1500 N/m, still more preferably about 400 N/m to about 1000 N/m, most preferably about 500 N/m to about 800 N/m.

In order to prolong battery life and reduce the possibility of false positive event identifications, the monitor may have a means for turning it off and on. In one embodiment, the monitor includes a simple on/off switch. In another embodiment, the monitor is turned on and off using commands from the display device. In a preferred embodiment, the monitor is powered off after a predetermined interval of inactivity, either by the display device, or preferably by the monitor itself. In a particularly preferred embodiment, the monitor incorporates a motion sensor such as an accelerometer, with dedicated, low power circuitry that is capable of powering on the balance of the monitor electronics when a motion is sensed, for example when the device to be monitored is picked up, and the monitor turns itself off based on a period of inactivity, the inactivity being determined based on a combination of such parameters as device motion, measured vibration amplitude, successful completion of event identification, successful transmission of data to the display device, etc. Preferably, in the embodiment that the device is turned on in response to movement of the device, the vibration sensor that senses motion of the device is the same vibration sensor that monitors the use of the device as described previously. Optionally, the monitor may include a light sensor, and turn on in response to changes in light level, for example when the device is removed from a case, pocket, purse, glove compartment, or the like. In another embodiment, the device stays powered on continuously, and has either sufficient battery life or the capability of battery recharge.

The monitor and/or display may incorporate a feedback system to guide the user to the correct delivery maneuver during the delivery event. For example, the patient may be presented with a green and red light on the monitor, or a similar green or red shape on the display. In one possible embodiment, when the patient is inhaling too slowly, the lights do not light up. When the patient inhales too rapidly, the red light flashes, indicating that the patient should inhale slower. A solid green light indicates a proper inhalation. Any number of other feedback methods, including but not limited to vibrations, graphical displays, light(s) or voice instructions, may be used. In one preferred embodiment, the patient is presented with a graphical display of flow rate on the display device. A preferred flow rate range is highlighted, for example by a different color or by a box. When the patient inhales through the inhaler, the flow rate is displayed on the graph or on a gauge like display similar to a speedometer. The flow rate may be displayed as a graph of flow rate vs. time, but preferably only the flow rate at the current time point is displayed during an inhalation, for example as a line, box, or arrow on the graph. In this way, the patient can immediately modify his or her inhalation flow rate by inhaling harder or softer until the indicated flow rate is within the preferred range. This feedback may be used for each delivery event. In a preferred embodiment, the feedback method is used during initial training. If the monitor system determines that a delivery event has occurred outside of a prescribed or desired range, the display may prompt the user to use the feedback method again for the next event. Preferably data related to the quality of the inhalation, and optionally a graph of the inhalation flow rate vs. time, is available to the patient and other after the inhalation maneuver is complete.

The monitor electronics may conduct many functions selected from a list including but not limited to vibration measurement, analysis, storage, wireless transmission, battery management and status, motion sensing, noise cancellation, timing, time and date stamping, power on and off, control of feedback features, storage of sample signals' time waveforms and/or frequency spectra and analysis parameters. These features may be implemented using discrete electronic circuits, but are preferably implemented using one or more integrated circuits. In a preferred embodiment, the electrical components consist essentially of a battery, one or more vibration sensors, one or more noise cancellation microphone transducers, and a single application specific integrated circuit containing an analog to digital converter, memory, a microprocessor and a means of data communication to the display device. In another preferred embodiment, the application specific integrated circuit is comprised of one or more vibration sensors and optionally one or more noise cancellation microphone transducers.

The data generated by the monitor and display device can be used in many ways, including but not limited to dosing reminders, compliance monitors, dose counters, feedback and/or training as to the proper use of the device to be monitored, determining the best way of using the device, drug usage diaries, dosing lock-outs, overdose warnings, alerts to the patient, caregiver, family, legal authorities, etc. The data may also be pooled with the data from other users.

Many drug delivery devices and formulations require manipulation such as shaking prior to delivery. Preferably the monitoring system of the current invention reminds the patient that manipulation is required, and then monitors and verifies that sufficient manipulation such as shaking has occurred. If the patient forgets or insufficiently applies the manipulation, the user can be notified of this fact, and/or reminded prior to one or more subsequent delivery events. In a preferred embodiment, the vibration sensor of the current invention is used to monitor the amount of manipulation.

Many drug delivery devices, especially dry powder inhalers, must be held in a prespecified orientation, such as level, after the dose is readied for delivery, in order that the dose stay in the proper location until delivered, so that the highly flowable powder does not migrate from the desired presentation to the inhalation air flow. The monitoring system of the current invention preferably monitors the orientation of the device, for example by means of analysis of the measured signals from the vibration sensor, which is preferably a DC coupled accelerometer, and prompts the user to hold the device in the correct orientation, and/or notifies the patient if the correct orientation is not maintained. The monitoring system preferably reminds the user as to the correct orientation if the correct orientation was not maintained during a previous delivery event. The monitoring system may optionally prompt the user to take an additional dose if it is determined that the orientation of the device would lead to under dose or no dose. The monitor of the current device preferably contains one or more single or multi-axis accelerometers that are used to monitor the orientation. Preferably one or more of the accelerometers that are used to monitor the orientation of the device to be monitored is the same accelerometer that is used to monitor device vibrations. In a preferred embodiment, the monitor contains a multi-axis accelerometer or one or more additional accelerometers that are at a fixed angle, preferably essentially at a right angle, to each other, and accelerometers are used to monitor device orientation. The multiple accelerometers may also be used to monitor device vibrations in independent directions, and the combined data used to identify events, measure parameters such as inhalation flow rate, and to reduce sensitivity to ambient acoustic sound pressure and vibration.

Some inhalation devices, such as Diskus, have a cover on the device airway or a mechanism that otherwise blocks airflow through the airway until the dose is ready for inhalation, for example by advancing the dose strip in Diskus. This creates the problem that training maneuvers can only be done after the dose has been readied. To address this issue, the monitoring system can do training in conjunction with a dosing event, and if the inhalation is not performed properly, the patient can be prompted to perform additional training inhalation maneuvers prior to closing the airway or preparing the next dose. In this embodiment of the invention, the monitor would check for the vibration signal's time waveforms and/or frequency spectra and/or analysis results thereof associated with preparing the dose, for example advancing the dose strip, and notify the patient not to perform the inhalation training maneuver, avoiding a potentially dangerous overdose. Similarly, the monitoring system can monitor for multiple doses being prepared prior to a dosing event, and prompt the user to take an action to avoid an overdosing event. By way of an example, if multiple strip advance maneuvers are completed with a Diskus device prior to an inhalation, the user can be prompted to put the Diskus in a mouthpiece down orientation, and tap the mouthpiece on a surface such as a table top, thereby clearing the multiple doses from the system. The user would then be prompted to prepare an additional dose and inhale as usual. In a preferred embodiment, the patient is notified of the potential overdose condition by an audio signal or alarm, either by the monitor itself, or by the display device. In that way the patient is alerted even if they are not looking at the display device.

In one embodiment of the invention, the monitor is supplied with a mechanism for exciting vibrations of the surface of the device to be monitored, this vibration is sensed by the accelerometer, and this signal's time waveform and/or frequency spectrum is used, for example, to verify proper installation of the monitor or to calibrate the sensing of the device vibrations. The vibrating mechanism may be separate from the vibration sensor, or it may be the vibration sensor. For example, the vibration sensor may be electrically induced to excite a vibration using a pulse or other waveform, and then the vibration sensor may be used to monitor the resulting vibrations. In another embodiment, the excitation device is also a speaker used for sending other alerts, for example tilt or potential overdose conditions.

In one embodiment of the invention, the monitor may be supplied with features such as a finger groove or grooves, and the user is instructed to hold the device to be monitored and attached monitor by placing a finger or fingers in the groove or grooves. In this way, the device is held in a repeatable way, and the device vibrations are repeatably affected or preferably not affected by the way the user holds the device. The grooves may contain one or more sensors or switches that can be used to monitor the correct placement of the finger or fingers. In a preferred embodiment, the finger placement sensor is also the switch that turns on the device, ensuring that the device is only turned on when held properly. Preferably the monitoring system provides feedback to the user that the monitor has been turned on via proper finger placement, for example with a sound—a light on the monitor, and/or an indication on the display device.

It is an object of the invention to supply a system for monitoring the use of a device to be monitored, which is preferably a drug delivery device.

It is a further object of the invention to supply a monitor which identifies the usage of a device based on a characteristic vibration of said usage.

It is a further object of the invention to supply a monitor which can be used with a device to be monitored which does not require any electrical, pneumatic, or mechanical interface or interference with functional or moving components such as fluid flow, triggering or actuation mechanisms of the device to be monitored.

It is a further object of the invention to supply a monitor that can be attached to a device to be monitored without requiring any disassembly and reassembly of the device to be monitored.

It is a further object of the invention to supply a device such as a drug delivery device wherein the vibration monitoring and optional analyzing capabilities are factory built into the device, and are optionally electronically integrated with other electrical or electronic functions of the device.

It is a further object of the invention to supply a monitoring system for an inhalation drug delivery device that is capable of recording drug delivery events, inhalation flow profiles, and associated inhalation parameters selected from a list including but not limited to inhalation flow rate, depth of inhalation, total inhaled volume, inhaled volume after the device is actuated, coordination of the inhalation and actuation of the device, inhalation rate and inhaled volume at the time of actuation, etc., without requiring modification of the device airflow paths by the inclusion of, for example, airway extensions, optical, pressure, or other sensors in fluid contact with the device airflow, or holes in the device airway walls, for example for pressure transducers or other sensors.

It is a further object of the invention to supply a means for determining the dose delivered from a drug delivery device by sensing the duration of the vibration made by the delivery.

It is a further object of the invention to supply a means for determining the dose delivered from a drug delivery device by counting the number of repeating vibrations the occur during the delivery, for example wherein each repeating vibration is associated with 1 IU or 0.5 IU of insulin or insulin analog delivered from an insulin pen.

It is a further object of the invention to supply a monitor with multiple accelerometers at fixed angles, preferably right angles, to each other.

It is an aspect of the invention to monitor device orientation using one or more vibration sensors such as direct coupled (DC) accelerometer(s).

It is an aspect of the invention to supply a means for exciting a vibration of a device to be monitored and then monitor the resulting vibrations, in order for example to verify proper installation of the monitor or to calibrate the monitoring of vibrations.

It is an aspect of the invention to ensure that the device to be monitored is held properly with features on the monitor for the placement of one or more fingers.

It is an aspect of the invention to ensure that the device to be monitored is held properly by way of a turn on switch that must be held on via a predetermined finger placement during use, for example drug delivery.

It is a further object of the invention to minimize the abuse potential of addictive or abused drugs by monitoring their usage and notifying predetermined people if the usage is outside of predetermined guidelines.

It is a further object of the invention to ensure that a patient keeps a parenteral delivery device in place until the delivery is complete

It is a further object of the invention to supply a monitor for a drug delivery or other medical device that is used to treat multiple patients, for example in mass vaccination campaigns or bio-terror response.

It is a further object of the invention to supply a means for displaying information such as the time and date of a delivery event, for example on a smartphone or tablet computer.

It is a further object of the invention to supply a monitoring system which can detect and identify an event such as the actuation of a device to be monitored based solely on the vibration of the event.

It is a further object of the invention to monitor for manipulations that indicate that multiple doses have been prepared for delivery prior to delivery, and prompt the user to take actions to avoid a potentially dangerous overdose.

It is a further object of the invention to supply a device which is capable of measuring inhalation parameters, including but not limited to inhalation flow rate, depth of inhalation, inhaled volume after the device is actuated, coordination of the inhalation and actuation of the device, inhalation at the time of actuation, based solely on the vibrations made by the actuation of an inhaler and the vibration made by the air flowing through the inhaler

It is a further object of the invention to supply a monitor that can be easily and quickly attached to a device to be monitored, for example by the user.

It is a further object of the invention to supply a vibration monitor for a device to be monitored that can be made relatively insensitive to the exact location of the monitor on the device to be monitored.

It is a further object of the invention to supply a method of calibrating a flow monitor for a device to be monitored that does not require any additional flow generation or measuring equipment.

It is a further object of the invention to supply a device which uses the vibration generated by inhalation through a device to control a means of giving feedback to the patient as to his or her inhalation maneuver during the drug delivery event.

It is a further object of the invention to supply skilled caregivers and facilities with a record of medical device usage to show that prescribed therapies and procedures were delivered in the way intended.

It is a further object of the invention to supply skilled caregivers and facilities with a record of when prescribed therapies and procedures were not delivered as intended

It is an object of the invention to reduce or eliminate the possibility of interference to the vibration signal's time waveforms and/or frequency spectra and/or analysis results thereof from ambient acoustic sound pressure sources.

It is an advantage of the invention that it has reduced likelihood of damaging or otherwise impacting the functionality of the device to be monitored during installation of the monitor and use.

It is an object of the invention to supply training to the user of a device to be monitored, and suggest that he or she repeat training if the device is subsequently used incorrectly.

It is an object of the invention to supply instructions during the use of a device to be monitored

It is an object of the invention to supply feedback to a user during a drug delivery event, guiding them to the proper use of the drug delivery event.

It is an object of the invention to minimize or eliminate the need to charge or change the batteries of a device to be monitored.

It is an object of the invention to supply a monitor that can be removably attached to the device to be monitored.

It is an object of the invention to supply a system which combines a drug delivery monitor with a monitor of disease state, and to create a single dataset containing data from both monitors.

It is an object of the invention to supply a system for pooling drug delivery usage data from many patients to thereby improve patient care.

It is an object of the invention to supply a system for documenting the degree to which patients in a pharmaceutical clinical trial are properly using a drug delivery device as prescribed.

It is an object of the invention to supply a single monitor design which can be used with multiple different devices to be monitored, for example different drug delivery technologies, thereby achieving better economies of scale and lower costs.

It is an object of the invention to supply a single monitor design, multiple examples of which can be used by an individual user with multiple different devices to be monitored, for example different drug delivery technologies.

It is an object of the invention to supply a single monitor design, multiple examples of which can be used by a single patient with multiple different inhalers, for example with prevention inhaler and a rescue inhaler.

It is an object of the invention to improve morbidity and mortality by ensuring that medications are delivery properly.

It is an object of the invention to reduce costs associated with untreated disease due to incorrect delivery or non-delivery of prescribed medications.

It is an advantage of the invention that there is reduced likelihood of interfering with the triggering, actuation, airflow, drug delivery, or aerosol generation of a drug delivery device, and therefore reduced risk of overdose, underdose, or no dose to the patient.

It is an advantage of the invention that it is less sensitive to the location of a vibration monitor for a device to be monitored.

It is an advantage of the invention that the monitor is easier to install on the device to be monitored.

It is an advantage of the invention that it can respond to slow changes in the measured vibration signal's time waveforms and/or frequency spectra and/or analysis results thereof due to wear of device components, depletion of formulation in the drug reservoir, residual drug left on device surfaces, etc.

An aspect of the invention is a monitoring system for use with a device to be monitored, comprising:
a display device;
a monitor comprising an vibration sensor;
an adhesive component for attaching the monitor to the device to be monitored;
a wireless transmitter for transmitting data from the monitor to the display device.

In another aspect of the invention the monitor is designed to be attached to the device to be monitored after the device to be monitored is fully assembled.

In another aspect of the invention the monitor is designed to be attached to a system that generates vibration when an event occurs, the monitor is designed to acquire and analyze samples of the vibration made during the event, and the monitoring system is designed to identify events based on a comparison to the analysis results from previously measured events.

In another aspect of the invention the attachment of the monitor does not require any disassembly of the fully assembled device.

In another aspect of the invention the monitor does not touch any moving elements of the device to be monitored.

In another aspect of the invention the device to be monitored is an inhaler, and monitor does not change the air flow path of the device.

In another aspect of the invention the adhesive comprises an carrier or an adhesive strip.

In another aspect of the invention the monitor is attached to a device to be monitored in a factory, doctor's office, or pharmacy.

In another aspect of the invention the monitor is attached after the device has been purchased.

In another aspect of the invention the monitor is attached by the end user.

In another aspect of the invention the monitoring system comprises a display device chosen from a smartphone, mp3 players, smartphones, Android phones, iPhones, Blackberry devices, Microsoft phones, eyeglasses capable of displaying information such as Google glass, a smart watch, a wearable device, tablet, notebook computer, desktop computer, television, DVD player, Blue-ray player, or streamer.

In another aspect of the invention the monitoring system comprises a software program.

In another aspect of the invention the monitoring system comprises a downloadable application.

In another aspect of the invention the monitoring system comprises a software package the operation of which can be customized based on a selected device to be monitored that the monitor is attached to.

In another aspect of the invention the monitoring system comprises a software package the operation of which can be customized based information supplied by the user.

In another aspect of the invention the monitoring system comprises a software package that can be customized based on parameters selected from a device to be monitored, a drug delivery device, a drug, a disease being treated, a state of the disease, properties of the patient selected from height, weight, sex, age, body mass index, race, medical conditions, pulmonary function parameters selected from peak flow, inspiratory flow rate, vital capacity, tidal volume, FEV1, FEVn, local weather conditions, ambient temperature, ambient pressure, one or more recipients for data sharing, opt in state for data sharing, physician, hospital, payer, provider, data sharing service, country.

In another aspect of the invention the monitoring system comprises a display which instructs a user or a caregiver as to the proper location for attaching the monitor on a selected device.

In another aspect of the invention the monitoring system comprises a display which instructs a user or a caregiver as to the proper procedure for attaching the monitor on a selected device.

In another aspect of the invention the monitoring system comprises a display for giving feedback to the user related to the correct use of the device to be monitored.

In another aspect of the invention the device to be monitored is a drug delivery device selected from an autoinjector, a needle-free injector, a bolus injector, and an infusion system, and further wherein the monitoring system comprises a display device which prompts the user maintain the placement of the device to be monitored until the drug delivery event is completed.

In another aspect of the invention the device to be monitored is an inhaler, and the monitoring system comprises a mechanism for giving feedback and training to the user during a delivery event selected from reminders based on errors made in previous dosing events, a reminder to shake the inhaler, a reminder to open the inhaler, a reminder to advance a dose strip, a reminder to insert a dosage form, a reminder to prime the device, a reminder to hold the device in a prescribed orientation such as level, a reminder to fully exhale prior to inhaling, target inhalation flow rate range, actual inhalation rate, a target inhaled volume, actual inhaled volume, when to trigger the inhaler, when to begin inhaling, when to stop inhaling, breath hold duration, a reminder to close the device.

In another aspect of the invention, the device is an inhaler, and the monitoring system comprises a mechanism for giving feedback to a user selected from verification that the inhaler was sufficiently shaken, verification that the inhaler was primed, verification that the inhaler was opened, verification a dose strip was advance, verification a dosage form was inserted, verification that an inhalation of sufficient depth was performed, verification that an inhalation of acceptable flow rate was performed, verification that the device was actuated at an acceptable point during the inhalation, verification that the inhaler was closed.

In another aspect of the invention the device to be monitored is an inhaler, and the monitoring system comprises a mechanism for giving feedback and training to the user following a delivery event selected from shaking the device, priming the device, advancing a dose strip, inserting a dosage form, inhaling through the device, triggering the device, closing the device.

In another aspect of the invention the device to be monitored is an inhaler, and the monitoring system comprises software that determines inhalation flow rate through the inhaler based on the vibration made during inhalation.

In another aspect of the invention the device to be monitored is an inhaler, and the monitoring system comprises software that computes inhalation flow rate through the inhaler based on measured properties of the vibration signal's time waveforms and/or one or more frequency spectra measured during inhalation.

In another aspect of the invention the device to be monitored is an inhaler, and the monitoring system comprises software that determines inhalation flow rate through the inhaler based on a comparison of the amount of vibration in two or more frequency bands and a comparison to a model of the vibration in the two or more frequency bands, said model being based on data previously generated using another example of the inhaler.

In another aspect of the invention the device to be monitored is an inhaler, and the monitoring system comprises software that determines inhalation flow rate through the inhaler based on properties of vibration made during inhalation and additional information selected from a model of vibration vs. inhalation rate for the inhaler, data from a previous inhalation through the device by the patient, data related to the patient selected from height, weight, sex, age, race, body mass index, vital capacity, peak inspiratory flow rate, inspired volume, tidal volume, FEV1, FEVn.

In another aspect of the invention the device to be monitored is a parenteral drug delivery device selected from an injector, an autoinjector, a needle free injector, a bolus injector, and an infusion pump

In another aspect of the invention the device to be monitored is a parenteral drug delivery device, and the monitoring system comprises a mechanism for giving feedback to a user selected from reminders to remove a cap or cover from the device, reminders to shake or otherwise agitate the device, reminders to prime the device, reminders to select a desired dose, reminders to press the device against a desired delivery site, reminders to insert a needle or catheter at a desired delivery site, reminders to actuate the device, reminders to leave the device in place until delivery is complete, reminders to remove the device from the delivery site, reminders to replace a cap or cover on the device.

In another aspect of the invention the device to be monitored is a parenteral drug delivery device, and the monitoring system comprises a mechanism for giving feedback to a user selected from verification that a cap or cover was removed from the device, verification that that the device was sufficiently shaken or otherwise agitated, verification that the device was primed, verification that a desired dose was selected, verification that the device was pressed against a desired delivery site, verification that a needle or catheter was inserted at a desired delivery site, verification that the device was actuated, verification that the delivery time was within an acceptable range, verification that the device was removed from the delivery site, verification that a cap or cover was replaced on the device.

In another aspect of the invention the device to be monitored is a parenteral drug delivery device, and the monitoring system comprises a mechanism for giving feedback and training to the user following a delivery event selected from removal of a cap or cover, shaking or otherwise agitating, priming, pressing against a desired delivery site, inserting a needle or catheter, actuating, delivering, removing the device from the delivery site, replacing a cap or cover.

In another aspect of the invention the monitoring system comprises a display device and software, wherein the display device must be in data communication with the monitor and running the software for the monitoring system to operate.

In another aspect of the invention the monitoring system comprises a display device and software, wherein the display device need not be in data communication with the monitor and running the software for the monitor to operate.

In another aspect of the invention the monitoring system comprises a display device and software, wherein the monitor is capable of identifying a drug delivery event without the display device being in data communication with the monitor and running the software, further wherein information related to the drug delivery event is transmitted to the display device at a later time when the display device is in data communication with the monitor and running the software.

In another aspect of the invention the monitoring system comprises a display device and software, wherein the monitor is capable of making a preliminary identification of a drug delivery event without the display device running the software, further wherein information related to the drug delivery event is transmitted to the display device at a later time when the display device is running the software, after which transmitting the software makes a final determination of the identification and quality of a drug delivery event.

In another aspect of the invention the monitor comprises software that allows the monitor to acquire a sample vibration signal's time waveform and/or frequency spectrum.

In another aspect of the invention the monitoring system comprises software that compares an acquired vibration signal's time waveform and/or frequency spectra and/or analysis results thereof to a previously stored reference time waveform and/or frequency spectrum and/or analysis results thereof to determine if a drug delivery event has occurred and the nature of the event.

In another aspect of the invention the monitor acquires a signal's time waveform and/or one or more frequency spectra based on preset criteria, and subsequently sends the signal's time waveform and/or frequency spectra to a display unit for further processing.

In another aspect of the invention the monitor comprises batteries which are rechargeable.

In another aspect of the invention the monitor comprises batteries which are replaceable.

In another aspect of the invention the monitor comprises batteries which are neither rechargeable nor replaceable.

In another aspect of the invention the monitor is attached essentially non-removably to the device to be monitored.

In another aspect of the invention, ambient acoustic sound pressure and/or vibration levels are monitored prior to a delivery event, and an action is taken if ambient acoustic sound pressure or vibration levels exceed a prespecified level.

In another aspect of the invention the monitor is attached removably to the device to be monitored.

In another aspect of the invention the monitor comprises an electronic component, and a separate component comprising elements selected from:
an adhesive,
a release liner,
a power source

a means for mechanically connecting the separate component to the monitor

In another aspect of the invention the monitor is supplied as a kit which comprises an electronic component, and a multiplicity of separate components comprising an adhesive, a release liner, and a mechanism for removably attaching one of the separate components to the electronic component, the attachment comprising at least one of: an electrical attachment, a mechanical attachment.

An aspect of the invention is a method, comprising:
downloading software to a display device;
running the software;
instructing a user to select a device to be monitored in the software
instructing the user as to the attachment of a monitor to the device to be monitored;
instructing the user as to the correct usage of the device to be monitored;
providing the user with feedback related to a usage event, preferably a dosing event.

It in an object of the invention to provide a monitoring system for a device to be monitored, comprising a vibration sensor that generates an electronic signal, wherein the sensor is selected from the list which comprises:

an accelerometer;

a vibration velocity sensor; and

a relative motion sensor;

wherein the monitoring system further comprises an electronic storage device holding stored electronic information corresponding to an expected sensor signal associated with a desired operation of the device to be monitored; and a software program which identifies an event based an analysis of the measured electronic signals from the vibration sensor by comparing the analysis results with the stored electronic information, wherein the device to be monitored is a drug delivery device.

It in an object of the invention to provide a display which displays information related to the identified event.

It in an object of the invent to provide a software program performs an operation based on one of:

the identification of the event; and

the determination that an event has not occurred;

wherein the operation is selected from the group consisting of:

presenting feedback relative to the quality of the event

presenting an instruction;

presenting a warning;

preventing a subsequent action;

enabling a subsequent action;

calculating an inhalation flow rate;

calculating an inhaled volume;

displaying an inhalation flow rate; and

calculating a delivered dose.

It in an object of the invent to provide a wireless data transmitter

It in an object of the invent to provide a display device comprising a display and a component selected from the list consisting of:

a wireless data receiver;

a microprocessor;

a speaker; and

a keypad.

It in an object of the invent to provide a display device is selected from the list consisting of:

a smartphone;

a smartwatch;

glasses;

a tablet; and

a laptop computer.

It in an object of the invention to provide a display which displays information selected from the list consisting of
instructions for installation of the monitor on the device to be monitored;
instructions for preparation for use of the device to be monitored;
instructions for use of the device to be monitored;
feedback related to a previous use of the device to be monitored;
information related to charge status of a battery;
information related to servicing of the device to be monitored;
information related to the type of device to be monitored; and
information related to the user of the device to be monitored.

It in an object of the invention to provide a display which allows entry of information selected from the list consisting of:
information related the prescribed use of the device to be monitored;
information related to servicing of the device to be monitored;
information related to the type of device to be monitored; and
information related to the user of the device to be monitored.

It in an object of the invention to provide a monitoring system which comprises a monitor which is essentially always powered on.

It in an object of the invention to provide a monitoring system comprised a monitor that is powered on based on an event selected from the list consisting of:
movement of the device to be monitored;
actuation of a switch;
output from a sensor;
receipt of a data transmission;
arrival of a predetermined time; and
elapse of a predetermined time interval.

It in an object of the invention to provide a monitor which is powered off based on an event selected from the list consisting of:
lack of movement of the device to be monitored;
actuation of a switch;
output from a sensor;
receipt of a data transmission;
arrival of a predetermined time; and
elapse of a predetermined time interval.

It in an object of the invention to provide a vibration sensing component comprised of the vibration sensor wherein the vibration sensing component is preloaded into contact with the device to be monitored by means of a compliant, compressed preload mechanism.

It in an object of the invention to provide a monitor comprising electronic components and the vibration sensor, and a carrier comprising an adhesive for attachment to the device to be monitored, and a mechanism for removably attaching the monitor to the carrier.

It in an object of the invention to provide a vibration sensing component which comprises a rigid component to which the vibration sensor is attached, wherein the carrier is comprised of a hole through which the rigid component contacts the device to be monitored.

It in an object of the invent to provide a vibration sensing component which is preloaded into contact with the device to be monitored by a force of from about 0.1 N to about 2 N.

It in an object of the invention to provide a carrier is essentially irremovably attached to the device to be monitored, and is disposed of with the device to be monitored.

It in an object of the invention to provide a monitor which is packaged with the device to be monitored at the point of sale.

It in an object of the invention to provide a vibration sensor which is essentially irremovably attached to the device to be monitored.

It in an object of the invention to provide a vibration sensor which is essentially irremovably attached to a component of the device to be monitored via a method chosen from the list consisting of:

adhering;

clamping;

taping;

screw threads;

a click fitting;

a bayonet fitting; and

a press fit.

It in an object of the invention to provide a vibration sensor is mounted on a circuit board, and the circuit board is mounted to the device to be monitored.

It in an object of the invention to provide a monitoring system as described herein, wherein the device to be monitored is a drug delivery device is selected from the group consisting of

an inhaler;

a pen injector;

a bolus injector; and

an autoinjector.

It in an object of the invention to provide a vibration sensor which is a direct coupled accelerometer, and the monitoring system responds to a tilt of the drug delivery device in a way selected from:
a visual warning; and
an audio warning.

preferably wherein the device to be monitored is a dry powder inhaler

It in an object of the invention to provide a monitoring system for an inhaler, and the monitoring system calculates an inhalation flow rate through the inhaler during an inhalation based on a computation selected from the list consisting of:

a vibration amplitude;

an RMS vibration;

a vibration in at least one preselected frequency band;

an offset measured before the start of the inhalation; and

an offset measured after the completion of the inhalation.

preferably wherein the computation is based on a portion of the inhalation of duration of 500 ms or less.

more preferably wherein the duration is 100 ms or less.

It in an object of the invention to provide a monitoring system which computes a dose delivered based on the duration of a vibration wave form.

It in an object of the invention to provide a monitoring system for a pen injector, and the monitoring system computes a dose delivered based on a count of repeating, similar vibration wave forms, preferably wherein the dose is comprised of one of;
insulin; and
an insulin analog.

It in an object of the invention to provide a monitoring system for a drug delivery device, comprising a vibration sensor that generates an electronic signal, wherein the sensor is selected from

an accelerometer,

a vibration velocity sensor,

a relative motions sensor,

wherein the monitoring system further comprises an electronic storage device holding stored electronic information corresponding to an expected vibration sensor signal associated with a desired operation; and a software program which identifies an event based an analysis of the measured electronic signals from the vibration sensor by comparing the analysis results with the stored electronic information, and an additional component selected from the list consisting of:

a display which displays information related to the identified events;

a wireless data transmitter;

a wireless data receiver; and

a microprocessor.

a speaker; and

a keypad.

These and other objects, advantages, and features of the invention will become apparent to those persons skilled in the art upon reading the details of the devices and methodology as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to scale. On the contrary, the dimensions of the various features are arbitrarily expanded or reduced for clarity. Included in the drawings are the following figures:

FIG. 1 shows one embodiment of the display of the invention

FIG. 2 shows one embodiment of the monitor and carrier of the invention

FIG. 3 shows one embodiment of the carrier and optional battery of the invention

FIG. 4 shows a side view the embodiment of the carrier and optional battery of FIG. 3

FIG. 5 shows an embodiment of the monitoring system of the current invention including a display device and monitor attached to an inhaler.

FIG. 6 shows an embodiment of the monitoring system of the current invention, with a display, a disease state monitor, and the monitor attached to an autoinjector.

FIG. 7 shows frequency spectra generated with the vibration sensor of the current invention when air is drawn at various flow rates through an inhalation device to be monitored (Diskus).

FIG. 8 shows the frequency spectra of FIG. 7 in the frequency bands of 3.15 kHz to 16 kHz.

FIG. 9 shows the rms vibration in the frequency bands of FIG. 8 combined as a function of flow rate through the inhalation device to be monitored.

FIGS. 10a, 10b, 10c, 10d and 10e each show a prototype of the invention designed for use with Diskus.

FIG. 11 shows a computed sample inhalation profile through diskus.

### DETAILED DESCRIPTION OF THE INVENTION

Before the present formulations and methods are described, it is to be understood that this invention is not limited to particular formulations and methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a formulation” includes a plurality of such formulations and reference to “the method” includes reference to one or more methods and equivalents thereof known to those skilled in the art, and so forth. In particular, “the spectrum” of a signal includes reference to multiple spectra that may be acquired during an event.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

An accelerometer is a device that can measure the rate of acceleration, whether caused by gravity or by movement. An accelerometer can therefore measure the rate of change of the speed of movement of an object it is attached to and that movement includes direct, mechanical vibrations of the object. An accelerometer may be comprised of piezoelectric sensors that can determine if a device is accelerating. Information is sent from the sensors and the system's program can convert this information into an accurate measure of the acceleration and the direction of acceleration.

Monitor: An electronic device that is capable of monitoring the vibration signals made by an event, optionally analyzing the monitored vibration signals, and transmitting information related to the event to a display device for display, further analysis, and further data transmission. The monitor includes a system for adhering it, removably, or non-removably, to a device to be monitored, preferably a drug delivery device. Preferably the transmitting is wireless.

Monitoring System: a system for monitoring the usage of a device to be monitored, preferably a drug delivery system, that has functions selected form providing instructions and/or suggestions to the user, storing, analyzing, and/or displaying data related to usage, sending alerts, monitoring disease states. The Monitoring System of the current invention comprises a vibration sensor for monitoring and characterizing events. The Monitoring System preferably comprises systems selected from a monitor, one or more processing systems, a data transmission system which may be wired but is preferably wireless, a display system, and an alerting system. In a preferred embodiment, the monitoring system comprises a monitor with vibration acquisition technology, processing and storage functionality, a wireless transmission system, and a mechanism for either removably or permanently attaching the monitor to a device to be monitored. In the preferred embodiment, the monitoring system also includes a display device that comprises a wireless transmission system, processing and storage functionality, display functionality, and alerting functionality. The monitoring system may also incorporate or be interfaced with a disease state monitoring system, including but not limited to a glucose meter or a pulmonary function meter.

Carrier, adhesive strip, and the like: a component that adheres a monitor to a drug delivery or other device. The adhesive strip may be a simple adhesive that adheres the monitor in an essentially non-removable manner. In another embodiment, the carrier is attached non-removably to the device, and the monitor is attached removably to the carrier. The carrier preferably comprises an adhesive region which is covered prior to use by a release liner. Preferably the carrier comprises a hole through which an elevated or protruding portion of the monitor is secured. The monitor is attached to the carrier by any suitable means, including but not limited to a press fit, a screw thread, or a bayonet fitting, preferably with a detent that secures the connection and gives the user feedback that the monitor is attached properly. The protruding portion comprises or is rigidly attached to a vibration sensor, and the carrier and hole are designed such that the protruding portion is in rigid contact with the surface of the device to be monitored.

Display device: A device capable of receiving data transmission, preferably wireless transmission, from a monitor, analyzing the transmitted data, and displaying information including but not limited to the data, analysis results, training and feedback information, warnings, and alerts. Display devices may include any device capable of supplying the above functionality. Display devices may be purpose built for the application, but are preferably devices that the user already has and/or could use for other purposes. Examples of display devices include but are not limited to smartphones, mp3 players, Android phones, iPhones, Blackberry devices, Microsoft phones, eyeglasses capable of displaying information such as Google glass, smart watches, and other wearable devices, tablets, notebook computers, desktop computers, televisions, DVD players, Blue-ray players, or video streamers. Preferred display devices are smartphones and tablet computers. It will be obvious to one skilled in the art that future devices will be developed that are capable of being used as the display device of the current invention.

Signal: a set of data containing a vibration's time waveform and/or one or more frequency spectra. Often signals are related to an event to be tracked by the monitoring system, such as a drug delivery event. Preferred signals include the vibration of a device to be monitored makes when it is loaded, readied for delivery, or triggered; acceleration due to gravity when a device is tilted; the vibration of the device created by air travelling through an inhaler when a user inhales through it; and/or the vibration or vibrations created by a pen injector, autoinjector, bolus injector, or pump during delivery.

Compliance monitor: a device that captures the time and date at which a device, preferably a drug delivery device, is used, preferably along with information related to the proper or improper use of the device.

Feedback: Information given to a user of a device, preferably a patient using a drug delivery device, related to their usage of the device. Feedback may be given while a dosing event is occurring, or may in the form of information and suggestions after the event or multiple events. Preferred feedback relates to inhalation flow rate and volume during a dosing event from an inhaler.

Vibration sensor, vibration sensor, and the like: A device which converts a mechanical vibration waveform of a solid body into a corresponding electrical waveform of essentially the same shape (over a range of frequencies) with an amplitude which is proportional to the amplitude of the vibration signal. Preferred vibration sensors include vibration velocity sensors, vibration relative motion sensors, and are most preferably accelerometers.

Vibration velocity sensor: a device that converts movement (velocity) of an attached object into voltage, which may be recorded. The most common type of vibration velocity sensors are geophones, which are passive analog devices and typically comprise a spring-mounted magnetic mass moving within a wire coil to generate an electrical signal.

Relative motion sensor: a device which detects the change in the relative position between two locations associated with mechanical movements and generates an electronic signal in response to detected relative motions. There are many varieties of contact vibration relative motion sensors such as LVDTs and non-contact vibration relative motion sensors based on optical, capacitive, inductive and other sensing techniques.

The terms event, dosing event, delivery event, and the like shall be interpreted to mean an occurrence which is instructed and/or monitored by the monitoring system of the current invention. Preferably the occurrence is the administration of drug to a patient in need thereof, preferably by a drug delivery device, which is preferably but not limited to the intrapulmonary or transdermal route of administration, infusion, or injection. Information related to dosing events is preferably acquired by a monitor and transmitted to a display device.

The term “inspiratory flow rate”, “inspiratory flow” and the like shall mean a value of the volume of air per unit time at a given time passing through an inhaler during a dosing event. Average inspiratory flow rate is the average over a predetermined fixed time, or preferably over an entire inhalation.

The term “inspiratory volume”, “inspired volume” and the like shall mean a measured, calculated and/or determined volume of air passing through an inhaler and into the lungs of a patient

The term “inspiratory flow profile” shall be interpreted to mean inspiratory flow rate data as a function of time as calculated during an inhalation delivery or training event. The profile preferably encompasses the entire inhalation.

The term “formulation” is used herein to describe any pharmaceutically active drug by itself or with a pharmaceutically acceptable carrier preferably in a flowable form which is preferably a liquid or powder. Liquid formulations are preferably solutions, e. g. aqueous solutions, ethanolic solutions, aqueous/ethanolic solutions, saline solutions and colloidal suspensions. Formulations can be solutions or suspensions of drug in a low boiling point propellant. Preferred formulations include liquids and powders for inhalation injection, transdermal administration, or infusion.

The terms “lung function” and “pulmonary function” are used interchangeably and shall be interpreted to mean physically or mechanically measurable operations of a lung including but not limited to (1) inspiratory and (2) expiratory flow rates as well as (3) lung volume. Methods of quantitatively determining pulmonary function are used to measure lung function. Quantitative determination of pulmonary function may be important when delivering analgesic drugs in that respiration can be hindered or stopped by the overdose of such drugs. Methods of measuring pulmonary function most commonly employed in clinical practice involve timed measurement of inspiratory and expiratory maneuvers to measure specific parameters. For example, forced vital capacity (FVC) measures the total volume in liters exhaled by a patient forcefully from a deep initial inspiration. This parameter, when evaluated in conjunction with the forced expired volume in one second (FEV1), allows bronchoconstriction to be quantitatively evaluated. A problem with forced vital capacity determination is that the forced vital capacity maneuver (i.e. forced exhalation from maximum inspiration to maximum expiration) is largely technique dependent. In other words, a given patient may produce different FVC values during a sequence of consecutive FVC maneuvers. The FEF 25-75 or forced expiratory flow determined over the midportion of a forced exhalation maneuver tends to be less technique dependent than the FVC. Similarly, the FEV1 tends to be less technique dependent than FVC Similarly to FEV1, FEVn is the forced expiratory volume in n seconds. In addition to measuring volumes of exhaled air as indices of pulmonary function, the flow in liters per minute measured over differing portions of the expiratory cycle can be useful in determining the status of a patient's pulmonary function. In particular, the peak expiratory flow, taken as the highest air flow rate in liters per minute during a forced 15 maximal exhalation, is well correlated with overall pulmonary function in a patient with asthma and other respiratory diseases. The present invention carries out treatment by administering drug in a drug delivery event and monitoring lung function in a monitoring event. A series of such events may be carried out and repeated over time to determine if lung function is improved. Each of the parameters discussed above is measured during quantitative spirometry. A patient's individual performance can be compared against his personal best data, individual indices can be compared with each other for an individual patient (e.g. FEV1 divided by FVC, producing a dimensionless index useful in assessing the severity of acute asthma symptoms), or each of these indices can be compared against an expected value. Expected values for indices derived from quantitative spirometry are calculated as a function of the patient's sex, height, weight and age. For instance, standards exist for the calculation of expected indices and these are frequently reported along with the actual parameters derived for an individual patient during a monitoring event such as a quantitative spirometry test.

The term “bolus injector” shall be interpreted to mean a wearable infusion device that delivers an infusion over a period of time which is longer than is typical for a injection device, for example longer than 1 minute, longer than 5 minutes, or longer than 20 minutes, but is shorter than is typical for a pump, for example less than 5 hours, less than 1 hour, or less than 30 minutes.

The term “autoinjector” shall be interpreted to mean a self contained injector that contains the formulation to be injected as well as a power source, trigger, and actuator for the injection. The formulation may be a replaceable single dose container or a multidose container. Autoinjectors may also be single use disposable, and may be factory prefilled with the formulation. Needle free injectors are an example of an autoinjector that utilizes a liquid jet of formulation to pierce the skin rather than a needle. Autoinjectors are preferable small, light and portable, for example small enough to be carried in a pocket, purse or automobile glove compartment.

The term “pump” shall be interpreted to mean a device that delivers drug, preferably subcutaneously or intravenously, via a catheter over a long period of time, typically longer than an hour, longer than 12 hours, or longer than 1 day. Pumps may be hospital based, for example pole mounted, or they may be wearable. Patch pumps are small self contained devices that adhere to the skin similarly to a transdermal patch, plaster, or adhesive bandage. Pumps may operate in a continuous infusion mode, a bolussing mode, or both. Pumps often deliver insulin, insulin analogs, or analgesics.

The term “pen injector” shall be interpreted to mean a device for delivering a drug, usually insulin or insulin analogs. Pen injectors are typically mechanical injectors with the approximate size and form factor of a ball point pen. Preferred pen injectors allow the patient to titrate the delivered amount of medication from a multi dose drug reservoir, typically in increments of 0.5 IU or 1.0 IU of insulin or insulin analogs. Typically pen injector have functionality that allows the patient to set the desired dose via a knob and a mechanical dose display, and contain a plunger that is depressed to deliver the medication. Preferred pen injectors create a repeatable vibration for each increment of dose that is delivered when the plunger is depressed.

The term “essentially irremovably attached” shall be interpreted to mean not designed to be detached and reused. The term shall not be interpreted to mean completely impossible to detach and reuse.

### DETAILED DESCRIPTION OF THE INVENTION

The current invention is a monitoring system for a device to be monitored, preferably a drug delivery device including but not limited to an inhaler, pen injector, autoinjector, that monitors the vibration made by the device when it is, for example, loaded or otherwise prepared, triggered, when the drug is delivered, or when an inhaler in inhaled through. The measured vibration signals' time waveforms and/or frequency spectra and/or analysis results thereof are preferably compared to pre-loaded reference time waveforms and/or frequency spectra and/or analysis results thereof and the match or comparison to these time waveforms and/or frequency spectra and/or analysis results thereof are used to identify a desired event, such as the loading or triggering of the device.

Measurement of the vibration of the device can be made by any vibration sensor, preferably by a vibration velocity sensor, vibration relative motion sensor, most preferably by an accelerometer. As used herein the term “vibration sensor” does not cover a microphone, which detects sound pressure waves rather than vibration.

An accelerometer is a device that measures proper acceleration (“g-force”). Proper acceleration is not the same as coordinate acceleration (rate of change of velocity). For example, an accelerometer at rest on the surface of the Earth will measure an acceleration g=9.81 m/s2 straight upwards. By contrast, accelerometers in free fall (for example orbiting) and coordinate accelerating due to the gravity of Earth, will measure zero acceleration.

Because an direct coupled (DC) accelerometer senses acceleration due to gravity, it can also sense the angle at which it is oriented, e.g. the angle at which it, and a device to be monitored to which it is attached, is being held. The movement and tilt of the device is noted by the sensors, so it can tell the angle at which the device is being held. This allows it to automatically adjust any output including visual output to make it appropriate to the direction of the device. In addition, the monitoring system can prompt the user to take certain actions if the device is held in an orientation that is detrimental to device operation. As an example, certain dry powder inhalers must be held in a prescribed orientation after the dose is readied, or the powder will flow to a location where it cannot be efficiently entrained in the inhalation airflow.

There are a wide range of accelerometers with multiple applications. Single-axis and multi-axis models of accelerometer are available to detect magnitude and direction of the proper acceleration (or g-force), as a vector quantity, and can be used to sense orientation (because direction of weight changes), coordinate acceleration (so long as it produces g-force or a change in g-force), vibration, shock, and falling in a resistive medium (a case where the proper acceleration changes, since it starts at zero, then increases). Micro-machined accelerometers are increasingly present in portable electronic devices and video game controllers, to detect the position of the device or provide for game input.

An accelerometer measures proper acceleration, which is the acceleration it experiences relative to freefall and is the acceleration felt by people and objects. Put another way, at any point in spacetime the equivalence principle guarantees the existence of a local inertial frame, and an accelerometer measures the acceleration relative to that frame. Such accelerations are popularly measured in terms of g-force.

An accelerometer at rest relative to the Earth's surface will indicate approximately 1 g upwards, because any point on the Earth's surface is accelerating upwards relative to the local inertial frame (the frame of a freely falling object near the surface). To obtain the acceleration due to motion with respect to the Earth, this “gravity offset” must be subtracted and corrections made for effects caused by the Earth's rotation relative to the inertial frame.

An accelerometer may comprise any

of, piezoelectric, piezoresistive and capacitive components commonly used to convert the mechanical motion into an electrical signal. Piezoelectric accelerometers rely on piezoceramics (e.g. lead zirconate titanate) or single crystals (e.g. quartz, tourmaline). They are unmatched in terms of their upper frequency range, low packaged weight and high temperature range. Piezoresistive accelerometers are preferred in high shock applications. Capacitive accelerometers typically use a silicon micro-machined sensing element. Their performance is superior in the low frequency range and they can be operated in servo mode to achieve high stability and linearity.

Modern accelerometers are often small micro electro-mechanical systems (MEMS), and are indeed the simplest MEMS devices possible, consisting of little more than a cantilever beam with a proof mass (also known as seismic mass). Damping results from the residual gas sealed in the device. As long as the Q-factor is not too low, damping does not result in a lower sensitivity.

Under the influence of external accelerations the proof mass deflects from its neutral position. This deflection is measured in an analog or digital manner Most commonly, the capacitance between a set of fixed beams and a set of beams attached to the proof mass is measured. This method is simple, reliable, and inexpensive. Integrating piezoresistors in the springs to detect spring deformation, and thus deflection, is a good alternative, although a few more process steps are needed during the fabrication sequence. For very high sensitivities quantum tunneling is also used; this requires a dedicated process making it very expensive. Optical measurement has been demonstrated on laboratory scale.

Another, far less common, type of MEMS-based accelerometer contains a small heater at the bottom of a very small dome, which heats the air inside the dome to cause it to rise. A thermocouple on the dome determines where the heated air reaches the dome and the deflection off the center is a measure of the acceleration applied to the sensor.

Most micromechanical accelerometers operate in-plane, that is, they are designed to be sensitive only to a direction in the plane of the die. By integrating two devices perpendicularly on a single die a two-axis accelerometer can be made. By adding an additional out-of-plane device three axes can be measured. Such a combination may have much lower misalignment error than three discrete models combined after packaging.

Micromechanical accelerometers are available in a wide variety of measuring ranges, reaching up to thousands of g's. The designer must make a compromise between sensitivity and the maximum acceleration that can be measured.

Accelerometers can be used to measure vibration on cars, machines, buildings, process control systems and safety installations. They can also be used to measure seismic activity, inclination, machine vibration, dynamic distance and speed with or without the influence of gravity. Applications for accelerometers that measure gravity, wherein an accelerometer is specifically configured for use in gravimetry, are called gravimeters.

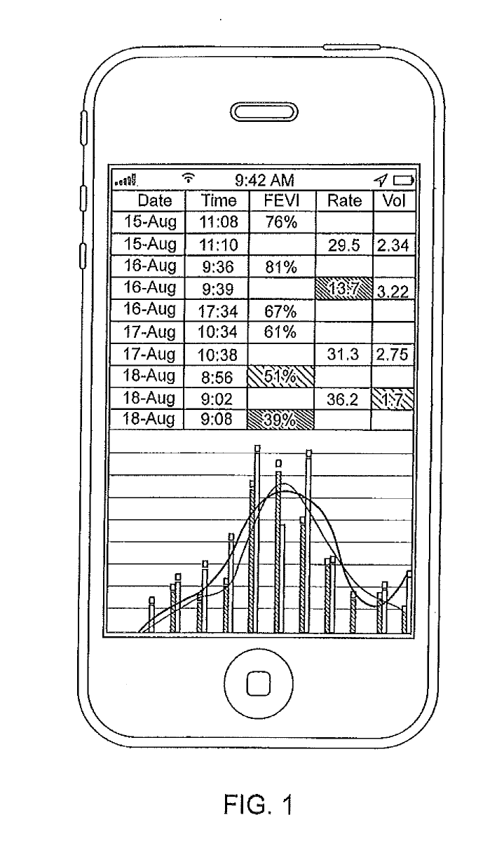

FIG. 1 shows an embodiment of display device 9 of the invention. As shown, display device 9 is a smart phone similar to an iPhone. Display device 9 can be any of a number of devices capable of displaying data and sending control commands to monitor 2, including but not limited to smartphones, mp3 players, Android phones, iPhones, Blackberry devices, Microsoft phones, eyeglasses capable of displaying information such as Google glass, smart watches, and other wearable devices, tablets, notebook computers, desktop computers, televisions, DVD players, Blue-ray players, or streamers. In a preferred embodiment of the invention, the display device is a smartphone or tablet computer.

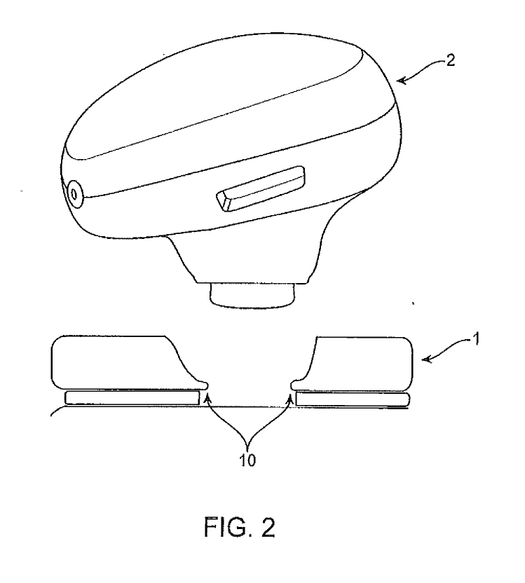

FIG. 2 shows an embodiment of the invention wherein monitor 2 is ready to be removably attached to carrier 1. Preferably, in this embodiment of the invention the user is supplied with a monitor 2 and a plurality of carriers 1. Prior to use, monitor 2 is attached to carrier 1 with a press fit, click attachment, screw attachment, or bayonet attachment. As shown in FIG. 2, monitor 2 clicks into place in carrier 1 via detents 10. Subsequently, carrier 1 is attached to the device to be monitored, preferably in a predetermined location, and preferably after removal of a release liner that exposes the adhesive. When the disposable device to be monitored or disposable component such as a drug cartridge of a durable device is expended, monitor 2 is removed from carrier 1, and carrier 1 is disposed of with the disposable device or drug cartridge.

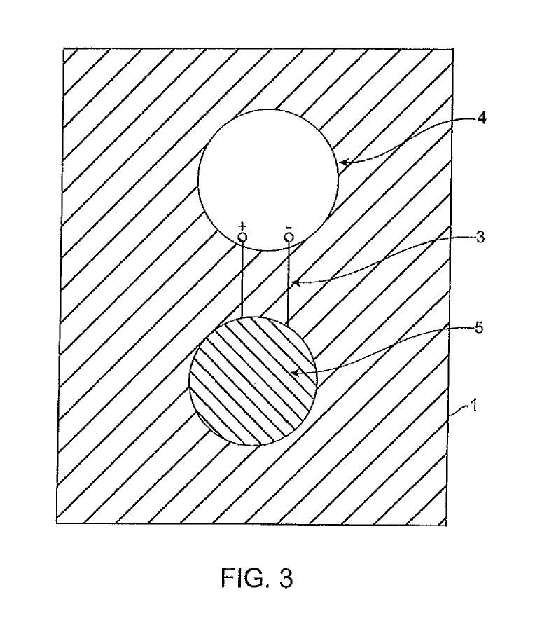

FIG. 3 shows a top view of an embodiment of carrier 1 prior to attachment to monitor 2. Carrier 1 optionally comprises energy source 5 such as an electrical cell or battery. Carrier 1 also has hole 4 into which a mating feature on monitor 2 is inserted. Hole 4 preferably comprises a through hole so that a protruding member that comprises or is rigidly attached to a vibration sensor and extends from the tip of the mating feature of monitor 2 can be brought into preloaded physical contact with the device to be monitored. Hole 4 optionally contains electrical contacts attached to electrical leads 3 for supplying electrical power to monitor 2. Hole 4 also contains detent features 10 to provide a positive attachment and click when the mating feature on monitor 2 is inserted.

FIG. 4 shows a side view of carrier 1. Substrate 8 supplies mechanical strength to carrier 1, and contains optional battery 5 and electrical leads 3. Substrate 8 may be rigid, or may be compliant for installation on profiled surfaces, for example the round surface of an insulin pen. Adhesive layer 6 is non-removably attached to substrate 8. Adhesive layer 6 may be thick and compliant enough to conform to non-planar surface profiles. Attached to adhesive layer 6 is removable release liner 7, shown in FIG. 4 partially removed. Hole 4 extends through substrate 8 and adhesive layer 6, but preferably not through release liner 7, so that it is obvious into which end of hole 4 the mating feature on monitor 2 should be inserted.

One embodiment of the use of the embodiment of FIGS. 1-4 is as follows. The system software, for example a smartphone or tablet application, is downloaded into display device 9, and the software is started. Display device 9 prompts the user to enter information selected from a list including but not limited to the device to be monitored, information including but not limited to usage frequency, expiration, disease state, prescribing physician, and user information such as age, height weight, body mass index, race, sex, agreement with sharing of data, etc. The following steps are conducted with instruction from display device 9.

Monitor 2 and one of carrier 1 are removed from their packaging, and a feature on monitor 2 containing the vibration sensor or spike attached thereto is inserted into the hole in carrier 1. Upon making of the electrical connection with leads 3 of carrier 1, monitor 2 powers up automatically, and automatically connects wirelessly with display device 9.

Display device 9 then instructs the user to remove the device to be monitored from its packaging, remove release liner 7 from carrier 1, and instructs the user as to the proper placement of carrier 1 on the device to be monitored.

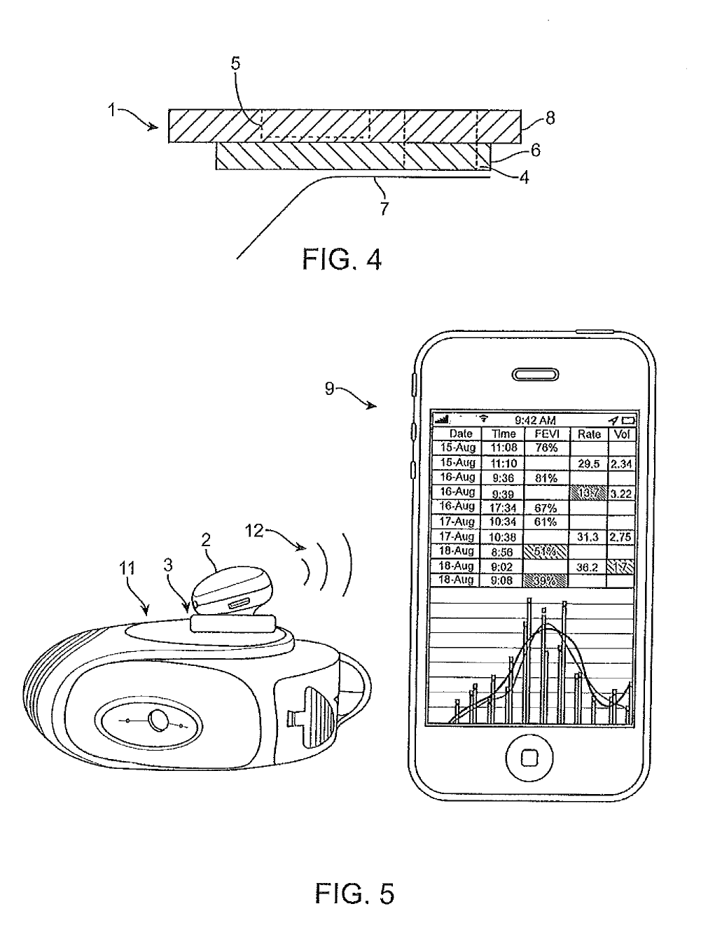

FIG. 5 shows the monitoring system of this embodiment at this stage, wherein monitor 2 is attached to inhalation device 11, and is transmitting wireless data 12 to display device 9.

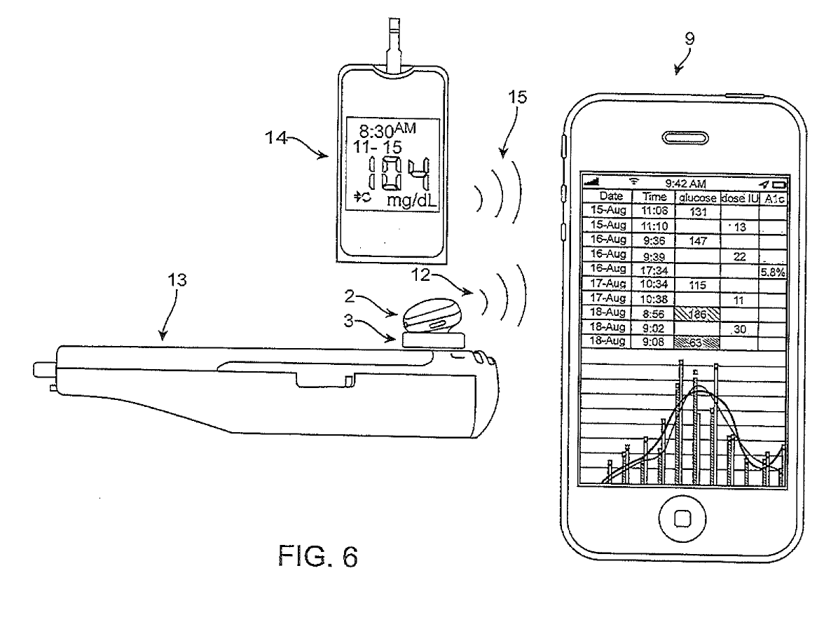

FIG. 6 shows the monitoring system of this embodiment attached to autoinjector 13. Also shown is optional blood glucose monitor 14 and wireless data from glucose monitor 14 to display device 9.

Optionally, display device 9 instructs the patient in the performance a maneuver, such as a dose delivery or inhalation through an inhaler, in order to train the system, calibrate the vibration signal's time waveform and/or frequency spectrum amplitude, verify functionality, etc.

The user is then instructed in the proper use of the device. Following a dosing event, display device 9 displays information related to the delivery event, and demonstrates to the user how to display information following future events.

Display device 9 continues to supply the user with additional information, for example dosing reminders, doses remaining, suggestions for improving delivery such as inhaling at a different rate or volume, and a record of all dosing events. Optionally display device 9 transmits the dosing data to, for example, an owner, a manufacturer, a security provider, a prescribing physician, a health maintenance organization, an insurance company, a court or police organization, a drug manufacturer, device to be monitored, or a data sharing, preferably a medical data web site or service provider.

When the device to be monitored is nearly depleted, expired, or otherwise requires servicing, for example when the drug reservoir of the drug delivery device is nearly depleted or the drug is nearly expired, display device 9 prompts the user to replace the device, drug cartridge, or other components or otherwise service the device. Monitor 2 is detached from carrier 1 by pulling in a direction perpendicular to substrate 8. Carrier 1 is disposed of with the device to be monitored or reservoir. A new carrier 1 and device to be monitored or reservoir are removed from their packaging, and the above steps are repeated.

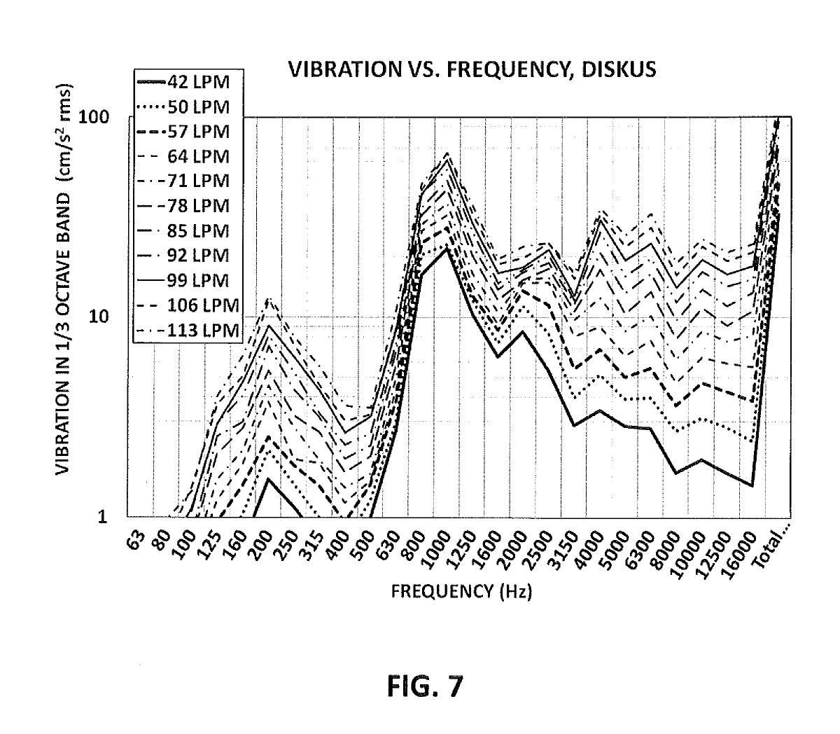

FIG. 7 shows the frequency spectra of the vibration of a Diskus device when air is drawn through it at various airflow rates. These data were generated with an accelerometer in contact with the exterior of case of a Diskus device in a sound isolated room. The data are displayed in ⅓ octave bands from 63 Hz to 16 kHz on a log scale. It can seen that the amplitudes of these frequency spectra increase with increasing flow rate over a wide range of frequencies and flow rates, showing that the current invention can use vibrations to determine flow rate through an inhalation device. It can be noted in FIG. 7 that there is a region near about 700 Hz where there is significantly less variation with flow rate than at higher frequencies. It may be possible to use the rms vibration in a frequency band in this region to correct for variation in overall amplitude, for example due to the details of the placement of the vibration sensor on the device that is somewhat independent of the inhalation flow rate. For example, the ratio of the rms amplitude in a band from about 4 kHz to about 16 kHz to the rms amplitude in a band that includes 700 Hz may be less sensitive to the location of the vibration sensor than simply using the rms amplitude in the 4-16 kHz band.

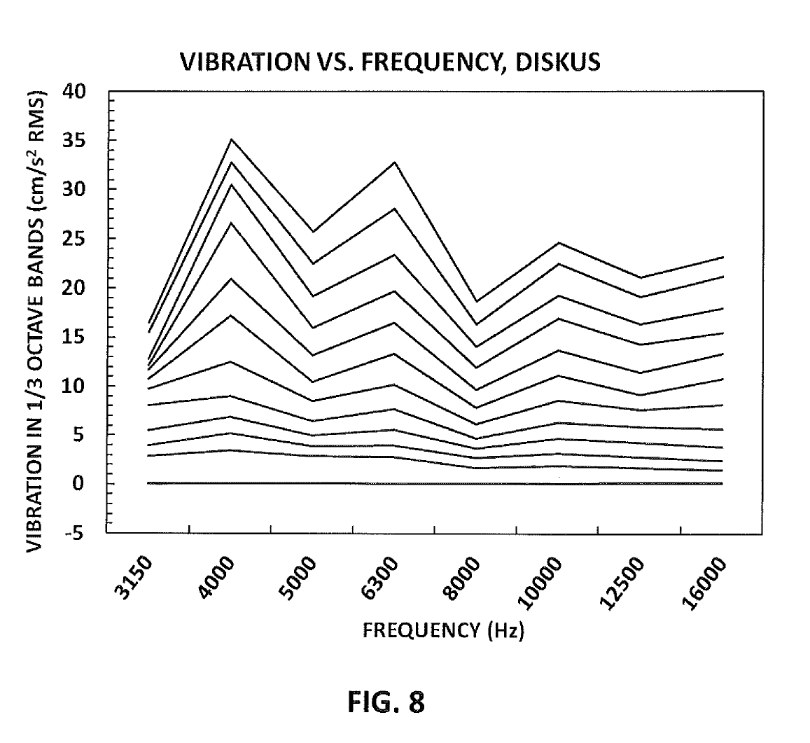

FIG. 8 shows the data of FIG. 7 in the ⅓ octave bands from 3.15 kHz to 16 kHz on a linear scale. This plot emphasizes the reproducible, steady increase in vibration in these frequency bands as the flow rate is increased from 42.45 Liters per Minute (LPM) to 113.20 LPM. Also included are data generated with the vacuum pump off and the vacuum pump on but the flow valve closed so there is zero air flow. These two lines, which essentially lie on top of each other at vibration=0, show that the source of the vibration is not ambient vibration or sound pressure, nor is the source of the vibration the vacuum pump.

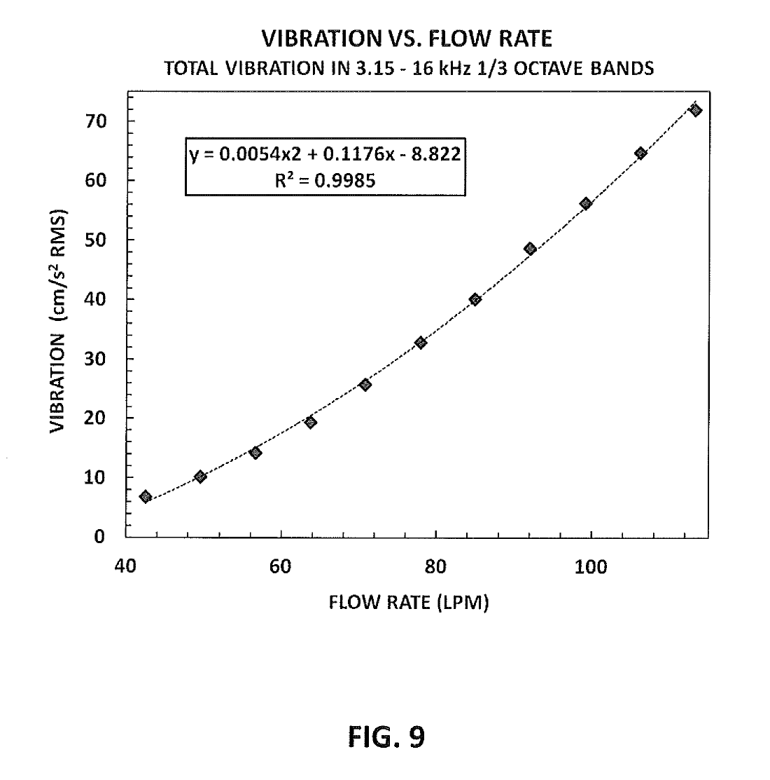

FIG. 9 shows the combined vibration (quadrature sum) of the vibration in the frequency bands of FIG. 8 vs. flow rate, along with a best fit quadratic function. These data demonstrate that the vibration sensor of the current invention can be used to accurately determine flow rate through a Diskus device.

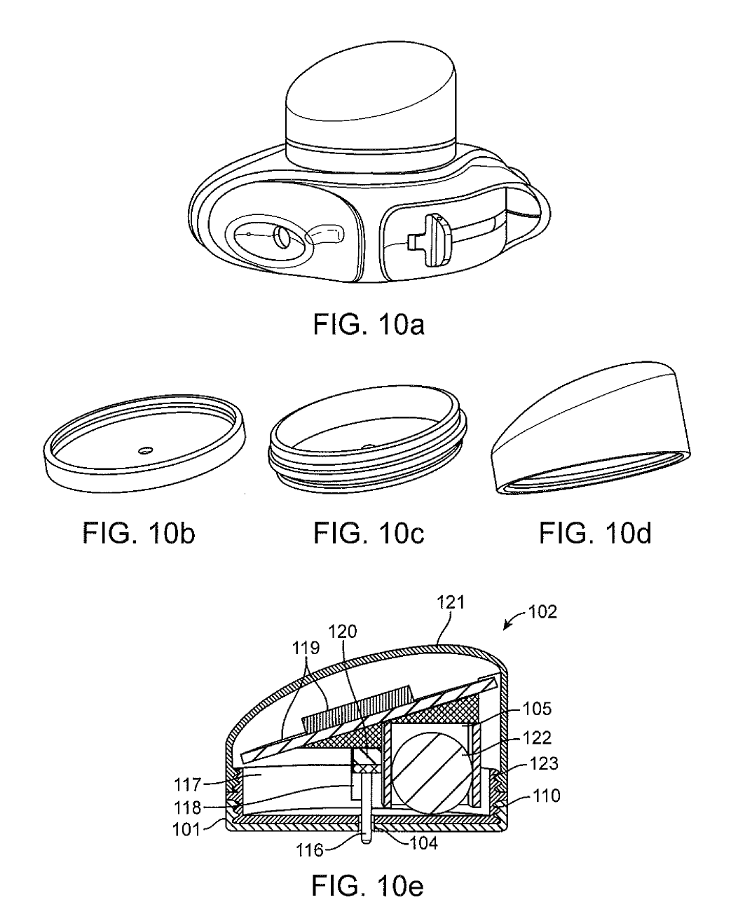

FIG. 10 shows a prototype of the device that was developed to demonstrate the monitoring of a Diskus device. FIG. 10a shows the fully assembled monitor 102 and carrier 101 attached to a Diskus device. FIGS. 10b-10c show the external plastic components, including cap 121 and adapter 117 that comprise the shell of monitor 102, and carrier 101. FIG. 10d shows a cross section view of fully assemble monitor 102 and carrier 101, including electronics and circuit board 119, spike 116 attached to accelerometer 118, compliant pre-load component 120, and battery 122 within battery housing 105. Spike 116 protrudes through hole 104 for contact with the device to be monitored (amount of protrusion exaggerated for clarity). Carrier 101 including the adhesive and release liner (not shown) are attached to adapter 117 via screw threads 110, and adapter 117 is attached to cap 121 via screw threads 123.

An embodiment of the use of the embodiment of FIG. 10 is as follows: Adapter 101 and the Diskus device are removed from their packaging. Following instructions from the display device, the release liner is removed from the adhesive on carrier 101, and carrier 101 is pressed onto the top of the Diskus device, aligning the outside diameter of carrier 101 with the raised circular detail on the top of the Diskus for proper placement. Fully assembled monitor 102 is then screwed into carrier 101 using screw threads 123. Spike 116 is pressed against the surface of the Diskus device, compressing compliant element 120 sufficiently to hold spike 116 rigidly against the top surface of the Diskus device. The rest of the usage is as described above relative to the embodiment of FIG. 5.

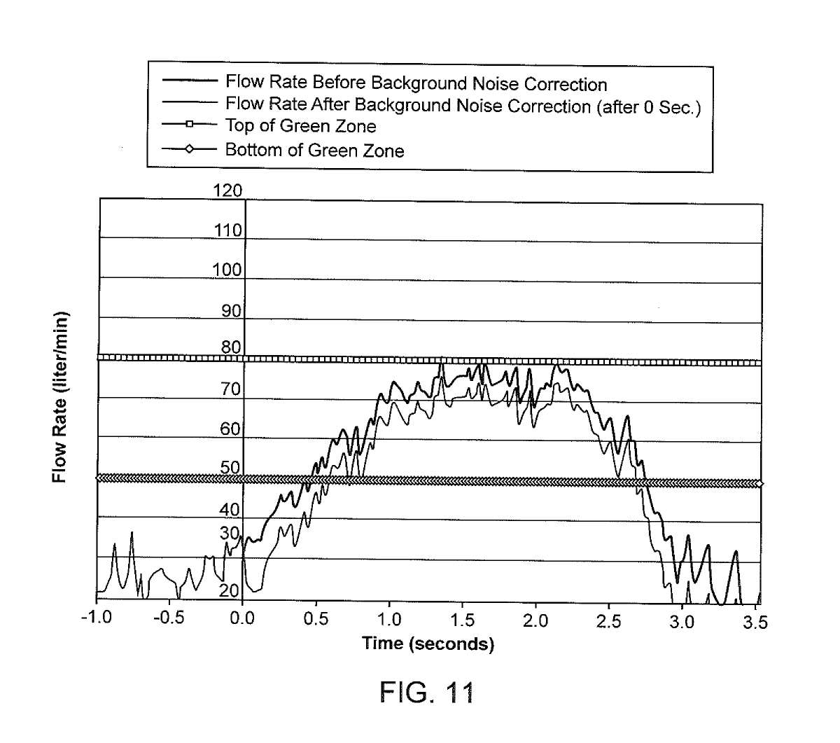

FIG. 11 shows an actual inhalation profile using the embodiment of FIG. 10 while inhaling through an attached Diskus device. The top curve shows the inhalation profile as computed based on the rms of the vibration wave form multiplied by a previously determined calibration factor. The curve prior to time t=0 shows the offset of the measured signal in the absence of inhalation flow. The lower curve after time t=0 show the calculated flow profile after removal of an average offset computed using the data prior to t=0.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

A physician has prescribed a long acting bronchodilator/inhaled corticosteroid dry powder inhaler product to a patient suffering from asthma, but the patient continues to have asthma attacks. The physician suggests the use of the monitoring system of the current device, and supplies the patient with the results of a recent pulmonary function test for vital capacity.

The patient purchases the monitor and 12 included carriers from the local pharmacy. Following the directions supplied with the monitor, the patients downloads an associated application to her smartphone, and runs the application.

The application prompts the patient to enter the type of inhaler being used, and her vital capacity. The patient, based on prompts from the smartphone application, removes a carrier from its packaging, removes a release liner from the carrier, removes a new inhaler from its packaging, and applies the carrier to a location on the inhaler as shown by a picture and associated instructions displayed by the smartphone application. Again following prompting by the smartphone application, the patient pairs the monitor to the smartphone using the Bluetooth functionality of the phone.

Following instructions from the smartphone application, the patient prepares the inhaler for delivery by removing a cap, and advancing a dose strip. Forgetting that she is supposed to hold the inhaler level, she tilts the inhaler and is given visual and auditory reminders to hold the inhaler level, which she does.

Following prompting from the smartphone, the patient exhales as fully as possible, puts the inhaler in her mouth, and inhales as deeply as possible. The monitor recognizes the characteristic vibration of inhalation through the device based on criteria wirelessly uploaded by the smartphone application, and wirelessly sends the accelerometer data to the smartphone.

Based on the assumption that the patient inhaled to her vital capacity, and using the vibration signal's time waveform and/or one or more frequency spectra of the inhalation and laboratory data related to the vibration generated by the inhaler as a function of inhalation flow rate, and the fact that the integrated flow rate over the duration of the inhalation must equal her vital capacity, the smartphone application calculates a calibration of flow rate vs. vibration amplitude specific to this particular monitor as installed on this device. The inhaler and monitor are now ready to use.

When the patient next doses using the device following a reminder from her phone to do so, she is notified via a test message that she inhaled too slowly, and did not inhale for a sufficient duration to get the entire dose. The application suggests a deeper, faster inhalation, and suggests that she look at the application running on the smartphone the next time she is using the inhaler.

The next day, the patient turns on her smartphone and opens the application prior to using her inhaler. The application recognizes vibrations that are characteristic of removing the cap and advancing the dose strip to the next dose, gives her feedback that these steps were performed correctly, and automatically displays a screen that is a graphical representation of inhalation flow rate, with a highlighted target range for flow rate, and a reminder to exhale fully before inhaling through the device. When the patient starts inhaling, she finds she can keep the inhalation rate in the target zone, and receives verbal reminders from the application to continue inhaling. When her inhalation is completed, she is presented with a breath hold countdown timer. She then receives feedback that her inhalation was done correctly.

Later that day she uses the smartphone application again, and again is able to achieve a successful delivery. The following day, she feels she can complete the inhalation maneuver without the feedback screen, and does not use the smartphone. She does not receive a notification that there was an issue with the inhalation. Curious, she looks at the log and it shows the most recent inhalation as successful.

She continues dosing with the device without thinking about the application. About two weeks later, she receives a notification that she needs to inhale more deeply. She opens the log in the smartphone application, and it shows that her inhaled volume had been slowly decreasing. The next day, she uses the application feedback function during her inhalation, and thereafter receives no additional notifications of an incorrect inhalation while using that device.

During the third week, she receives a notification that she has forgotten to take her dose, and takes the dose at the next convenient time.

When there are only 5 doses left in her inhaler, she receives a notice that she needs a new one. She calls the pharmacy, and the next day picks up her prescription refill.

### Example 2

A prototype monitor of the current invention was fabricated and tested. An ADXL335BCPZ (Analog Devices) low power, 3-axis±3 g accelerometer was used for vibration monitoring. The signal from the accelerometer was acquired and transmitted using a WT32i-A-ai6 (BlueGiga Technologies Inc.) Bluetooth module. A custom printed circuit board was developed for the Bluetooth module, on/off switch, and associated electronic components. Voltage for the accelerometer was supplied by a ML-621S/ZTN (Panasonic) 3V lithium battery, and power for the electronics was sourced by a 5HXF8 3V lithium battery. The accelerometer was rigidly attached to a custom pin, which was preloaded using a custom compliant elastomeric component fabricated from an ultra-soft 0.188″ silicone foam (BF-2000, Rogers corporation) with a spring constant of 640 N/m. A plastic case was designed and fabricated, and a carrier was developed for attachment to a “Diskus” (GSK) inhaler (see FIG. 10).

Analysis software was developed for an iPhone 6plus (Apple). The software allowed entry of patient data, selection of device to be monitored (only Diskus was selectable) and data sharing preferences. Instructions were developed for installation of the monitor on the Diskus device, and for use of the Diskus device. Opening of the Diskus device, advancement of the dose strip, and closing of the device were included in the instruction, and were monitored using the associated vibrations.

Diskus case vibrations were measured at air flow rates through the Diskus device from 42 to 113 LPM (FIG. 7). A calibration based on total RMS vibration measured vs. inhalation flow rate was developed, including an algorithm for background vibration signal subtraction. FIG. 11 shows a sample inhalation through the device, with and without background subtraction.

Additional information was supplied to the user through the iPhone application, including doses remaining and time to next dose. Prior to an inhalation, the user was given feedback from the previous delivery event, and if applicable, suggestions for improvement. During inhalation, the user was presented with a bar that moved up and down on the screen based on inhalation flow rate, and a target region for a correct inhalation. After inhalation, a 10 second breath hold count down was displayed. After the breath hold, the user was given feedback on the quality of the inhalation, the volume and rate of the inhalation, and a graph of the inhalation similar to FIG. 12. Feedback included “the inhalation was good”, “the inhalation was too slow, next time inhale harder”, “the inhalation was too fast, next time inhale slower”, “the inhalation was to shallow, next time inhale deeper”, and “the inhalation was too variable, next time inhale more steadily”. Previous inhalation data and graphs were available from a separate data screen.

After the inhalation and breath hold, but before the user closed the device, the user was given the option of entering a user training mode. The training mode is only available at this time, because prior to the strip being advanced, the airway is blocked by the Diskus mechanism to give the user feedback that the dose is not yet presented for delivery. Thus this is the only time the device was in a configuration where the user can inhale normally but not drug will be delivered. If the user selected the training mode, they were presented with an inhalation screen, breath hold timer, and data and feedback screen identical to an actual drug inhalation.

From the time the device was opened and ready for strip advance to the completion of the inhalation, the user was presented with a visible indication if they tilted the device past approximately 20 degrees from level.

In informal tests with the device, it was found that the instructions made it easy to use device correctly, and the feedback screen made it quite straight forward to perform an inhalation of the desired rate and depth.

The instant invention is shown and described herein in a manner which is considered to be the most practical and preferred embodiments. It is recognized, however, that departures may be made therefrom which are within the scope of the invention and that obvious modifications will occur to one skilled in the art upon reading this disclosure.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A monitoring system for a device to be monitored, comprising:
a vibration sensor that generates an electronic signal, wherein the sensor is selected from the list which consists of:
an accelerometer;
a vibration velocity sensor; and
a relative motion sensor;
wherein the monitoring system further comprises an electronic storage device holding stored electronic information corresponding to an expected sensor signal associated with a desired operation of the device to be monitored; and
a software program which identifies an event based an analysis of the measured electronic signals from the vibration sensor by comparing the analysis results with the stored electronic information,
a display which displays information related to the identified event,
a wireless data transmitter,
wherein the device to be monitored is a drug delivery device.

2. The monitoring system of claim 1, wherein the software program performs an operation based on one of:
the identification of the event; and
the determination that an event has not occurred;
wherein the operation is selected from the group consisting of:
presenting feedback relative to the quality of the event presenting an instruction;
presenting a warning;
preventing a subsequent action;
enabling a subsequent action;
calculating an inhalation flow rate;
calculating an inhaled volume;
displaying an inhalation flow rate; and
calculating a delivered dose.

3. The monitoring system of claim 2, wherein the wireless data transmitter is a Bluetooth transmitter.

4. The monitoring system of claim 2, wherein the display device is selected from the list consisting of:
a smartphone;
a smartwatch;
glasses;
a tablet; and
a laptop computer.

5. The monitoring system of claim 2, wherein the display device displays information selected from the list consisting of
instructions for installation of the monitor on the device to be monitored;
instructions for preparation for use of the device to be monitored;
instructions for use of the device to be monitored;
feedback related to a previous use of the device to be monitored;
information related to charge status of a battery;
information related to servicing of the device to be monitored;
information related to the type of device to be monitored; and
information related to the user of the device to be monitored.

6. The monitoring system of claim 5, wherein the display device allows entry of information selected from the list consisting of:
information related the prescribed use of the device to be monitored;
information related to servicing of the device to be monitored;
information related to the type of device to be monitored; and
information related to the user of the device to be monitored.

7. The monitoring system of claim 5, wherein the monitoring system comprised a monitor that is powered on based on an event selected from the list consisting of:
movement of the device to be monitored;
actuation of a switch;
output from a sensor;
receipt of a data transmission;
arrival of a predetermined time; and
elapse of a predetermined time interval.
further wherein the monitor is powered off based on an event selected from the list consisting of:
lack of movement of the device to be monitored;
actuation of a switch;
output from a sensor;
receipt of a data transmission;
arrival of a predetermined time; and
elapse of a predetermined time interval.

8. The monitoring system of claim 2, further comprising a vibration sensing component comprised of the vibration sensor,
wherein the vibration sensing component is preloaded into contact with the device to be monitored by means of a compliant, compressed preload mechanism.

9. The monitoring system of claim 8, comprising a monitor comprising electronic components and the vibration sensor, and a carrier comprising an adhesive for attachment to the device to be monitored, and mechanism for removably attaching the monitor to the carrier.

10. The monitoring system of claim 9, wherein the vibration sensing component comprises a rigid component to which the vibration sensor is attached, wherein the carrier is comprised of a hole through which the rigid component contacts the device to be monitored.

11. The monitoring system of claim 10, wherein the vibration sensing component is preloaded into contact with the device to be monitored by a force of from about 0.1 N to about 2 N.

12. The monitoring system of claim 9, wherein the carrier is essentially irremovably attached to the device to be monitored, and is disposed of with the device to be monitored.

13. The monitoring system of claim 5 wherein the vibration sensor is packaged with the device to be monitored at the point of sale.

14. The monitoring system of claim 13, wherein the vibration sensor is essentially irremovably attached to the device to be monitored via a method chosen from the list consisting of:
adhering;
clamping;
taping;
screw threads;
a click fitting;
a bayonet fitting; and
a press fit.

15. The monitoring system of claim 13, wherein the vibration sensor is mounted on a circuit board, and the circuit board is mounted to the device to be monitored.

16. The monitoring system of claim 5, wherein the drug delivery device is selected from the group consisting of:
an inhaler;
a pen injector;
a bolus injector; and
an autoinjector.

17. The monitoring system of claim 16, wherein the device to be monitored is an inhaler, and further wherein the vibration sensor is a direct coupled accelerometer, and the monitoring system responds to a tilt of the drug delivery device in a way selected from:
a visual warning; and
an audio warning.

18. The monitoring system of claim 16, wherein the drug delivery device is an inhaler, and the monitoring system calculates an inhalation flow rate through the inhaler during an inhalation based on a computation selected from the list consisting of:
a vibration amplitude;
an RMS vibration;
a vibration in at least one preselected frequency band;
an offset measured before the start of the inhalation; and
an offset measured after the completion of the inhalation,
wherein the computation is based on a portion of the inhalation of duration of 500 ms or less.

19. The monitoring system of claim 18, wherein the duration is 100 ms or less.

20. The monitoring system of claim 16, wherein the monitoring system computes a dose delivered based on one of:
the duration of a vibration wave form;
a count of repeating, similar vibration wave forms.

21. The monitoring system of claim 20, wherein the drug delivery device is a pen injector, the monitoring system computes the dose delivered based on a count of repeating, similar vibration wave forms, and the dose is comprised of one of;
insulin; and
an insulin analog.
